(19) United States
(12) Patent Application Publication
(10) Pub. No.: US 2005/0239155 A1
(43) Pub. Date: Oct. 27, 2005

(54) Entrapped binding protein as biosensors

(76) Inventors: Javier Alarcon, Durham, NC (US); J. Bruce Pitner, Durham, NC (US); Wensheng Cai, Durham, NC (US)

Correspondence Address: DAVID W. HIGHET;BECTON, DICKINSON AND COMPANY, 1 BECTON DRIVE, MC110, FRANKLIN LAKES, NJ, 07417, US

(21) Appl. No.: 11/077,028

(22) Filed: Mar. 11, 2005

Related U.S. Application Data
(63) Continuation-in-part of application No. 10/949,557, filed on Sep. 27, 2004.
Application No. 10/949,557 is a continuation of application No. 10/039,833, filed on Jan. 4, 2002, now abandoned.
(60) Provisional application No. 60/564,977, filed on Apr. 26, 2004.

(51) Int. Cl. C12Q 1/54; C12M 1/34

(52) U.S. Cl. 435/14; 435/287.1

(57) ABSTRACT

The invention is directed to binding proteins, proteins comprising reporter groups, compositions of binding molecules comprising reporter groups in analyte permeable matrices, and their use as analyte biosensors both in vitro and in vivo.

This application is a continuation-in-part of co-pending application Ser. No. 10/949,557, which is a continuation of application Ser. No. 10/039,833, filed Jan. 4, 2002, each of which is hereby incorporated by reference. This application also claims priority to U.S. provisional application Ser. No. 60/564,977, filed Apr. 26, 2004, which is hereby incorporated by reference.

### BACKGROUND OF THE INVENTION

1. Field of the Invention

The invention is in the field of biotechnology. Specifically, the invention is directed to binding molecules such as binding proteins, proteins comprising reporter groups, compositions of binding proteins comprising reporter groups in analyte permeable matrices, and their use as analyte biosensors both in vitro and in vivo.

2. Background of the Invention

Monitoring glucose concentrations to facilitate adequate metabolic control in diabetics is a desirable goal and would enhance the lives of many individuals. Currently, most diabetics use the “finger stick” method to monitor their blood glucose levels and patient compliance is problematic due to pain caused by frequent (several times per day) sticks. As a consequence, there have been efforts to develop non-invasive or minimally invasive in vivo and more efficient in vitro methods for frequent and/or continuous monitoring of blood glucose or other glucose-containing biological fluids. Some of the most promising of these methods involve the use of a biosensor. Biosensors are devices capable of providing specific quantitative or semi-quantitative analytical information using a biological recognition element.

The biological recognition element of a biosensor determines the selectivity, so that only the compound which has to be measured leads to a signal. The selection may be based on biochemical recognition of the ligand where the chemical structure of the ligand ( e.g. glucose) is unchanged, or biocatalysis in which the element catalyzes a biochemical reaction of the analyte.

The transducer translates the recognition of the biological recognition element into a semi-quantitative or quantitative signal. Possible transducer technologies are optical, electrochemical, acoustical/mechanical or colorimetrical. The optical properties that have been exploited include absorbance, fluorescence/phosphorescence, bio/chemiluminescence, reflectance, light scattering and refractive index. Conventional reporter groups such as fluorescent compounds may be used, or alternatively, there is the opportunity for direct optical detection, without the need for a label.

Biosensors specifically designed for glucose detection that use biological elements for signal transduction typically use electrochemical or colorimetric detection of glucose oxidase activity. This method is associated with difficulties including the influence of oxygen levels, inhibitors in the blood and problems with electrodes. In addition, detection results in consumption of the analyte that can cause difficulties when measuring low glucose concentrations.

A rapidly advancing area of biosensor development is the use of fluorescently labeled periplasmic binding proteins (PBP's). As reported by Cass (Anal. Chem. 1994, 66, 3840-3847), a labeled maltose binding protein (MBP) was effectively demonstrated as a useable maltose sensor. In this work MBP, which has no native cysteine residues, was mutated to provide a protein with a single cysteine residue at a position at 337 (S337C). This mutation position was within the binding cleft where maltose binding occurred and therefore experienced a large environmental change upon maltose binding. Numerous fluorophores were studied, some either blocked ligand binding or interfered with the conformational change of the protein. Of those studied, IANBD resulted in a substantial increase in fluorescence (160%) intensity upon maltose binding. This result may be consistent with the location of the fluorophore changing from a hydrophilic or solvent exposed environment to a more hydrophobic environment as would have been theoretically predicted for the closing of the hinge upon maltose binding. However, this mutant protein and the associated reporter group do not bind diagnostically important sugars in mammalian bodily fluids. Cass also disclosed Analytical Chemistry 1998, 70(23), 5111-5113 association of this protein onto TiO2 surfaces, however, the surface-bound protein suffered from reduced activity with time and required constant hydration.

Hellinga, et al. (U.S. Pat. No. 6,277,627), reports the engineering of a glucose biosensor by introducing a fluorescent transducer into a Galactose/Glucose Binding Protein (GGBP) mutated to contain a cysteine residue, taking advantage of the large conformation changes that occur upon glucose binding. Hellinga et al (U.S. Pat. No. 6,277,627) disclose that the transmission of conformational changes in mutated GGBPs can be exploited to construct integrated signal transduction functions that convert a glucose binding event into a change in fluorescence via an allosteric coupling mechanism. The fluorescent transduction functions are reported to interfere minimally with the intrinsic binding properties of the sugar binding pocket in GGBP.

In order to accurately determine glucose concentration in biological solutions such as blood, interstitial fluids, ocular solutions or perspiration, etc., it may be desirable to adjust the binding constant of the sensing molecule of a biosensor so as to match the physiological and/or pathological operating range of the biological solution of interest. Without the appropriate binding constant, a signal may be out of range for a particular physiological and/or pathological concentration. Additionally, biosensors may be configured using more than one protein, each with a different binding constant, to provide accurate measurements over a wide range of glucose concentrations as disclosed by Lakowicz (U.S. Pat. No. 6,197,534).

Despite the usefulness of mutated GGBPs, few of these proteins have been designed and examined, either with or without reporter groups. Specific mutations of sites and/or attachment of certain reporter groups may act to modify a binding constant in an unpredictable way. Additionally, a biosensor containing reporter groups may have a desirable binding constant, but not result in an easily detectable signal upon analyte binding. Some of the overriding factors that determine sensitivity of a particular reporter probe attached to a particular protein for the detection of a specific analyte are the nature of the specific interactions between the selected probe and amino acid residues of the protein. It is not currently possible to predict these interactions within proteins using existing computational methods, nor is it possible to employ rational design methodology to optimize the choice of reporter probes. It is currently not possible to predict the effect on either the binding constant or the selectivity based on the position of any reporter group, or amino acid substitution in the protein (or visa-versa).

To develop reagentless, self-contained, and or implantable and or reusable biosensors using proteins the transduction element must be in communication with a detection device to interrogate the signal to and from the transduction element. Typical methods include placing proteins within or onto the surface of optical fibers or planner waveguides using immobilization strategies. Such immobilization strategies include, but are not limited to, entrapment of the protein within semi-permeable membranes, organic polymer matrices, or inorganic polymer matrices. The immobilization strategy ultimately may determine the performance of the working biosensor. Prior art details numerous problems associated with the immobilization of biological molecules. For example, many proteins undergo irreversible conformational changes, denaturing, and loss of biochemical activity. Immobilized proteins can exist in a large number of possible orientations on any particular surface, for example, with some proteins oriented such that their active sites are exposed whereas others may be oriented such that there active sites are not exposed, and thus not able to undergo selective binding reactions with the analyte. Immobilized proteins are also subject to time-dependent denaturing, denaturing during immobilization, and leaching of the entrapped protein subsequent to immobilization. Therefore problems result including an inability to maintain calibration of the sensing device and signal drift. In general, binding proteins require orientational control to enable their use, thus physical absorption and random or bulk covalent surface attachment or immobilization strategies as taught in the literature generally are not successful.

There have been several reports of encapsulating proteins and other biological systems into simple inorganic silicon matrices formed by a low temperature sol-gel processing methods, for example, as taught by Brennan, J. D. Journal of Fluorescence 1999, 9(4), 295-312, and Flora, K.; Brennan, J. D. Analytical Chemistry 1998, 70(21), 4505-4513. Some sol-gel matrices are optically transparency, making them useful for the development of chemical and bio-chemical sensors that rely on optical transduction, for example absorption or fluorescence spectroscopic methods. However, entrapped or immobilized binding proteins must remain able to undergo at least some analyte induced conformational change. Conformational motions of binding proteins may be substantially restricted in most sol-gel matrices as taught in the literature. It has been reported that sol-gel entrapped proteins can exhibit dramatically altered binding constants, or binding constants that change over relatively short time periods or under varying environmental conditions. In addition, a time dependence of the protein function while entrapped in the sol-gel matrix has been reported. This time dependence of protein function in sol-gel entrapped matrices has limited general applicability of sol-gels in biosensors for in vitro as well as in vivo use.

Therefore, there is a need in the art to design additional useful mutated proteins and mutated GGBP proteins generating detectable signals upon analyte binding for use as biosensors, and additionally there is a need for the entrapment of these proteins into analyte-permeable matrices for interfacing to signal transmitting and receiving elements.

### BRIEF DESCRIPTION OF THE DRAWINGS

FIG. 1 illustrates the change in fluorescence response to glucose of A21 3C/L238C NBD amide GGBP H₆ in solution.

FIG. 2 illustrates signal enhancement of entrapped binding proteins in the absence and presence of analyte relative to solution.

FIG. 3 illustrates an entrapped binding protein in the absence and presence of analyte relative to solution.

FIG. 4 illustrates reversible signal from an entrapped binding protein from one embodiment of the present invention.

FIG. 5 depicts a schematic representation of a crosslinked poly(ethylene glycol) (PEG) hydrogel, with covalently attached binding molecules.

FIG. 6 depicts a graph demonstrating the fluorescence signal resulting from exposure of a PEG hydrogel glucose biosensor to 100 mM glucose.

FIG. 7 depicts a graph demonstrating that the fluorescence intensity of the PEG hydrogel glucose biosensor corresponds proportionally to the concentrations of glucose.

FIG. 8 depicts a graph demonstrating the stability of the PEG hydrogel glucose biosensors described herein, over a 40 day period.

FIG. 9 depicts a graph showing the response time of a PEG hydrogel glucose biosensor of the present invention in response to varying concentrations of ligand.

FIG. 10 depicts a graph tracking the changes in glucose concentrations in vivo in a pig using the PEG hydrogel glucose biosensors of the present invention.

FIG. 11 depicts a graph showing the response to varying concentrations of fatty acid by a PEG hydrogel fatty acid biosensor of the present invention.

FIG. 12 depicts the binding curve of a maltose biosensor comprising a maltose binding protein (labeled with IANBD) in a hydrogel, prepared by the “one pot” method.

FIG. 13 depicts the emission spectra of a maltose biosensor comprising a maltose binding protein (labeled with IANBD) in a hydrogel prepared by the “one pot” method in response to a solution of 100 mM maltose.

FIG. 14 depicts the binding curve of a maltose biosensor comprising a maltose binding protein (labeled with IANBD) in a hydrogel, prepared by the “two pot” method.

FIG. 15 depicts the ability of a maltose biosensor, comprising a maltose binding protein (labeled with IANBD) in a hydrogel located in an optical fiber, to track changes in maltose levels.

FIG. 16 depicts the response of a HEMA-MAA hydrogel glucose biosensor in the presence and absence of 100 mM glucose concentration.

FIG. 17 depicts the response of a HEMA-MAA hydrogel glucose biosensor at different glucose concentrations.

FIG. 18 depicts the glucose response of a poly(HEMA) hydrogel glucose biosensor coated on a 0.47 mm optical fiber

FIG. 19 depicts the changes in fluorescence intensity of hydrogel glucose biosensors in response to changing blood glucose levels in the glucose-controlled anesthetized pig.

FIG. 20 depicts the fluorescence response (QF) of hydrogel glucose biosensors prepared by the photo crosslinking methods described herein.

### SUMMARY OF THE INVENTION

The invention provides compositions comprising binding molecules in biosensors. In one specific embodiment, the invention provides a glucose biosensor including (a) a mutated binding protein and at least one reporter group attached thereto such that said reporter group provides detectable signal when said mutated binding protein is exposed to glucose and (b) a matrix permeable to analyte where the mutated glucose/galactose binding protein and the reporter group are entrapped within the matrix.

The invention also provides compositions comprising a mixture including (a) at least one binding protein and at least one reporter group attached thereto and (b) a hydrogel, dialysis membrane, sol-gel, or combinations thereof to provide for a matrix permeable to analyte wherein the binding protein and the reporter group are entrapped within the matrix.

In another specific embodiment, the invention also provides a composition and device including (a) a mutated maltose binding protein (MBP) and at least one reporter group attached thereto such that the reporter group provides a detectable signal when the mutated MBP is bound to maltose and wherein the MBP includes a cysteine present at position 337 and (b) a matrix permeable to maltose wherein the mutated MBP and the reporter group are entrapped within the matrix.

The invention further provides a device and compositions thereof suitable for in vivo use including (a) a mutated glucose/galactose binding protein and at least one reporter group attached thereto such that the reporter group provides a detectable and reversible signal when the mutated glucose/galactose binding protein is exposed to varying glucose concentrations and (b) a matrix permeable to analyte wherein the mutated glucose/galactose binding protein and the reporter group are entrapped within the matrix.

The present invention also relates to a biosensor comprising a polymeric hydrogel and a binding molecule. In one embodiment, the binding molecule is covalently attached to the hydrogel, and the binding molecule must be capable of generating a detectable signal upon target binding. In one particular embodiment, the biosensor is a glucose biosensor and comprises poly(ethylene glycol) and a glucose binding protein covalently attached thereto. In another particular embodiment, the biosensor is a glucose biosensor and comprises copolymers of hydroxyethylmethacrylate and methacrylic acid and a glucose binding protein covalently attached thereto. The invention also relates to methods of making and using biosensors.

### DETAILED DESCRIPTION OF THE INVENTION

The term biosensor generally refers to a device that uses specific biochemical reactions mediated by isolated enzymes, immunosystems, tissues, organelles or whole cells to detect chemical compounds, usually by electrical, thermal or optical signals. The compositions of the present invention must be able to function as biosensors. As used herein, “biosensor” is used to mean a composition, device or product that provides information regarding the local biological environment in which the product, device or composition is located. As used herein, a “biological environment” is used to mean an in vivo, in situ or in vitro setting comprising or capable of supporting tissue, cells, organs, body fluids, single-celled organisms, multicellular organisms, or portions thereof. The cells, tissue, organs or organisms, etc. or portions thereof can be alive (metabolically active) or dead (metabolically inactive). Examples of biological settings include, but are not limited to, in vitro cell culture settings, in vivo settings in or on an organism (such as an implant), a diagnostic or treatment setting, tool or machine, such as a DNA microarray or blood in a dialysis machine. The type of biological environment in which the biosensor can be placed should not limit the present invention.

The compositions of the present invention comprise a binding molecule. As used herein, a binding molecule is a molecule that binds to a ligand or complementary binding partner in a specific manner. A binding molecule can be a protein, such as a receptor, enzyme, antibody (or fragment thereof), or other binding protein. The binding molecule can also be a polynucleotide that can bind to other polynucleotides. Provided that the binding molecules bind in a specific manner to their target ligand, other examples of target ligands may include, but are not limited to, monosaccharides, disaccharides, polysaccharides, amino acids, oligopeptides, polypeptides, proteoglycans, glycoprotein, nucleic acids, oligonucleotides, lipids, fatty acids, natural or synthetic polymers, and small molecular weight compounds such as drugs or drug candidates.

In one embodiment, the binding molecule within the compositions of the present invention is a binding protein. The term “binding proteins” generally refers to proteins which interact with specific analytes in a manner capable of transducing or providing a detectable and or reversible signal differentiable either from when analyte is not present, analyte is present in varying concentrations over time, or in a concentration-dependent manner, by means of the methods described. The transduction event includes continuous, programmed, and episodic means, including one-time or reusable applications. Reversible signal transduction may be instantaneous or may be time-dependent providing a correlation with the presence or concentration of analyte is established. Examples of binding proteins include, but are not limited to, periplasmic binding proteins such as galactose/glucose binding protein (GGBP), maltose binding protein (MBP), ribose binding protein (RBP), arabinose binding protein (ABP), dipeptide binding protein (DPBP), glutamine binding protein (QBP), iron binding protein (FeBP), histidine binding protein (HBP), phosphate binding protein (PhosBP), and oligopeptide binding protein (OppA) or derivatives thereof. Another example of a binding molecule is fatty acid binding protein (FABP) or derivatives thereof.

“Binding proteins” generally refers herein to a family of proteins naturally found in the periplasmic compartment of bacteria. These proteins are normally involved in chemotaxis and transport of small molecules (e.g., sugars, amino acids, and small peptides) into the cytoplasm. For example, GGBP is a single chain protein consisting of two globular α/β domains that are connected by three strands to form a hinge. The binding site is located in the cleft between the two domains. When glucose enters the binding site, GGBP undergoes a conformational change, centered at the hinge, which brings the two domains together and entraps glucose in the binding site. X-ray crystallographic structures have been determined for the closed form of GGBP from E. coli (N. K. Vyas, M. N. Vyas, F. A. Quiocho Science 1988, 242, 1290-1295) and S. Typhimurium (S. L. Mowbray, R. D. Smith, L. B. Cole Receptor 1990, 1, 41-54) and are available from the Protein Data Bank (http://www.rcsb.org/pdb/) as 2GBP and 3GBP, respectively. The wild type E. coli GGBP DNA and amino acid sequence can be found at www.ncbi.nlm.nih.gov/entrez/accession number D90885 (genomic clone) and accession number 23052 (amino acid sequence). The GGBP may be from E. coli.

The binding proteins may be wild-type (native), or they may be a non-wild-type protein, provided that the proteins still bind to a target ligand in a specific manner. As used herein, a “non-wild-type protein” is a protein that shares substantial sequence identity with the wild-type protein. Examples of non-wild-type proteins include, but are not limited to, mutant and fusion proteins. “Mutated binding protein” (for example “mutated GGBP”) as used herein refers to binding proteins from bacteria containing amino acid(s) which have been substituted for, deleted from, or added to the amino acid(s) present in naturally occurring protein. The mutant binding proteins may be mutated to bind more than one ligand in a specific manner. Indeed, the mutant binding proteins may possess specificity towards its wild-type ligand and another target ligand.

Exemplary mutations of binding proteins include the addition or substitution of cysteine groups, non-naturally occurring amino acids (Turcatti, et al. J. Bio. Chem. 1996 271, 33, 19991-19998) and replacement of substantially non-reactive amino acids with reactive amino acids to provide for the covalent attachment of electrochemical or photo-responsive reporter groups.

The mutations in the non-wild-type binding proteins may serve one or more of several purposes. For example, a naturally occurring protein may be mutated in order to change the long-term stability of the protein; to conjugate the protein to a particular entrapment matrix, polymer; or to provide binding sites for detectable reporter groups, or to adjust its binding constant with respect to a particular analyte, or combinations thereof.

Exemplary mutations of the GGBP protein include a cysteine substituted for a lysine at position 11(K11C), a cysteine substituted for aspartic acid at position 14 (D14C), a cysteine substituted for valine at position 19 (V19C), a cysteine substituted for asparagine at position 43 (N43C), a cysteine substituted for a glycine at position 74 (G74C), a cysteine substituted for a tyrosine at position 107 (Y107C), a cysteine substituted for threonine at position 110 (T110C), a cysteine substituted for serine at position 112 (S112C), a double mutant including a cysteine substituted for a serine at position 112 and serine substituted for an leucine at position 238(S1 12C/L238S), a cysteine substituted for a lysine at position 113 (K113C), a cysteine substituted for a lysine at position 137 (K137C), a cysteine substituted for glutamic acid at position 149 (E149C), a double mutant including a cysteine substituted for an glutamic acid at position 149 and a serine substituted for leucine at position 238 (E149C/L238S), a double mutant comprising a cysteine substituted for histidine at position 152 and a cysteine substituted for methionine at position 182 (H152C/M182C), a double mutant including a serine substituted for an alanine at position 213 and a cysteine substituted for a histidine at position 152 (H152C/A213S), a cysteine substituted for an methionine at position 182 (M182C), a cysteine substituted for an alanine at position 213 (A213C), a double mutant including a cysteine substituted for an alanine at position 213 and a cysteine substituted for an leucine at position 238 (A213C/L238C), a cysteine substituted for an methionine at position 216 (M216C), a cysteine substituted for aspartic acid at position 236 (D236C), a cysteine substituted for an leucine at position 238 (L238C) a cysteine substituted for a aspartic acid at position 287 (D287C), a cysteine substituted for an arginine at position 292 (R292C), a cysteine substituted for a valine at position 296 (V296C), a triple mutant including a cysteine substituted for an glutamic acid at position 149 and a alanine substituted for a serine at position 213 and a serine substituted for leucine at position 238 (E149C/A213S/L238S), a triple mutant including a cysteine substituted for an glutamic acid at position 149 and a alanine substituted for an arginine at position 213 and a serine substituted for leucine at position 238 (E149C/A213R/L238S). An exemplary mutant MBP includes, but is not limited to MBP-S337C.

The invention also contemplates that the mutant binding proteins may be able to only bind a ligand or ligands that the wild-type binding protein does not bind. Methods of generating mutant proteins, in general, are well-known in the art. For example, Looger, et al., (Nature 423 (6936): 185-190 (2003)), which is hereby incorporated by reference, disclose methods for re-designing binding sites within periplasmic binding proteins that provide new ligand-binding properties for the proteins. These mutant binding proteins retain the ability to undergo conformational change, which can produce a directly generated signal upon ligand-binding. By introducing between 5 and 17 amino acid changes, Looger, et al. constructed several mutant proteins, each with new selectivities for TNT (trinitrotoluene), L-lactate, or serotonin. For example, Looger et al. generated L-lactate binding proteins from ABP, GGBP, RBP, HBP and QBP. These and other mutant binding proteins could be attached to the matrices of the present invention, such as hydrogels, to prepare a biosensor specific for the target ligands to which the proteins bind, and are within the scope of the present invention.

Other examples of non-wild-type proteins that can be used in the preparation of the biosensors of the present invention include fusion proteins. A fusion protein is used herein as it is in the art, and methods of generating fusion proteins are well-known in the art. For example, fusion protein derivatives of binding proteins may include fusions of binding proteins with fluorescent proteins such as green fluorescent protein (GFP) or dsRed. In particular, fusion proteins that can be used in this present invention are described in pending U.S. application Ser. No. 10/721,091, filed Nov. 26, 2003, the entirety of which is hereby incorporated by reference. Other fusion proteins contemplated for use in the present invention may be engineered or mutated to have a histidine tag on the protein's N-terminus, C-terminus, or both termini. Histidine fusion proteins are widely used in the molecular biology field to aid in the purification of proteins. Exemplary tagging systems produce proteins with a tag containing about six histidines, with such tagging not compromising the binding activity of the binding protein.

In another embodiment of the present invention, the binding molecule, e.g., binding proteins, are functionally derivatized prior to, or simultaneously with, their incorporation into the matrix, such as a hydrogel. As used herein the term “functionally derivatized” is used to mean a molecule such as a protein, polypeptide or oligopeptide that has been modified with the addition of a polymerizable reactive group such that the functionally derivatized molecule can act as a monomer during the polymerization of the matrix, e.g., a hydrogel. Accordingly, one embodiment of the current invention relates to methods of making a biosensor, with the methods comprising polymerizing functionally derivatized binding proteins with one or more monomers to produce a crosslinked polymeric hydrogel biosensor. This method of forming the biosensor by polymerizing a functionally derivatized binding protein with monomer constituents of the hydrogel is, in essence, one particular embodiment of a method of covalently attaching a binding protein to the matrix, such as a hydrogel. Examples of polymerizable reactive groups that can be used to form a functionally derivatized binding molecule include, but are not limited to, glycidyl acrylate, N-acryloxysuccinimide (NAS), vinyl azlactone, acrylamidopropyl pyridyl disulfide, N-(acrylamidopropyl)maleimide, acrylamidodeoxy sorbitol activated with bisepoxide or bis-oxirane compounds, allylchloroformate, methacrylic anhydride, acrolein, allylsuccinic anhydride, citraconic anhydride, allyl glycidyl ether, or derivatives thereof. The functional derivatization may occur anywhere on the molecule that is amenable to accepting the reactive groups, such as a lysine or a cysteine residue on a polypeptide chain; and the functional derivatization may occur at one or more places on the protein or peptide chain. In one embodiment, a binding protein is functionally derivatized to comprise acrylate functional groups. Thus, the act of“functionally derivatizing” a protein would comprise adding, e.g., through a conjugation reaction, a polymerizable reactive group to a molecule, such as a protein, polypeptide, oligopeptide dipeptide or even a single amino acid. The invention also contemplates that non-wild-type proteins may also be functionally derivatized. A functionally derivatized protein may also be non-wild-type protein, as previously described herein, such as a mutant protein. In one specific embodiment, N-acryloyl succinimide (NAS) is reacted with lysine residues on a binding protein to provide an acrylate functionally derivatized binding protein described herein.

In the instant invention, analyte and binding molecule act as binding partners. The term “associates” or “binds” as used herein refers to specific binding. Affinity of specific binding can be assessed by calculating a relative binding constant such as, but not limited to, dissociation constant (Kd). The Kd may be calculated as the concentration of free analyte at which half the binding molecule is bound, or vice versa. When the analyte of interest is glucose, the Kd values for the binding partners are preferably between about 0.0001 mM to about 30 mM. Accordingly, the entrapped binding proteins of the present invention may be used in an in vitro or in vivo analyte assay which, for example, is capable of following the kinetics of biological reactions involving an analyte, such as glucose, as well as in clinical assays, and food or beverage industrial testing. Thus, in one embodiment of the current invention, the concentration of the binding protein in the matrix is less than the Kd of the protein in solution.

Likewise, one aspect of the present invention relates to methods of altering the affinity of the binding molecules towards their targets. In one embodiment, the present invention relates to methods of decreasing the affinity of a binding protein containing biosensor, as measured, for example, by the biosensor's apparent Kd, towards its target analyte. As used herein, “apparent Kd” is used to mean the overall measured dissociation constant of the biosensor towards an analyte, as assessed by the directly generated signal of the biosensor in response to the analyte. In another embodiment, the present invention relates to methods of altering the affinity of the binding molecule towards its target analyte, or altering the selectivity of a biosensor comprising binding molecule. For example, the entrapment of a GGBP within a matrix may decrease the affinity of the protein-containing biosensor towards glucose, thus increasing the apparent Kd of the biosensor, in relation to free GGBP in solution. Accordingly, one embodiment of the present invention relates to a biosensor comprising a binding molecule wherein the biosensor has an apparent Kd of at least about one order of magnitude greater than the Kd of the free binding molecule, i.e., a binding molecule not entrapped within or on a matrix, in solution. Similarly, a specific embodiment of the present invention includes biosensors for either glucose or maltose detection, with apparent Kd of at least one order of magnitude greater than the Kd of free GGBP or MBP, respectively.

The biosensors comprised of a matrix and a binding molecule must be capable of providing a detectable signal upon target ligand binding to the binding molecule. To “provide a detectable signal”, as used herein refers to the ability to recognize a change in a property of a reporter group within the biosensor in a manner that enables the detection of ligand-protein binding. In one embodiment, therefore, the binding molecules may comprise one or more “reporter groups,” e.g., a fluorescent protein or dye, that are responsible for generating the detectable signal which is altered upon a change in binding molecule conformation or reporter group environment which occurs, for example, upon analyte binding. Thus, in one embodiment of providing a detectable signal, the biosensor will generate a signal directly upon binding of the target ligand to the binding molecule. As used herein, “generating a signal directly upon binding” is used to mean that the act of binding of the analyte to the binding molecule itself is responsible for generating the detectable signal, without any additional reactions or processes. Furthermore, it is intended that a directly generated signal is a signal that is produced by the reporter group itself and not the matrix, e.g. hydrogel. A directly generated signal is not meant to include a signal that is generated from a chemical reaction that produces a product or byproduct which would then be measured, nor is a directly generated signal used to mean a signal that is generated from competitive binding to a labeled analyte, as disclosed in Russell, R. J. and Pishko, M. V., “A fluorescence-based glucose biosensor using concanavalin A and Dextran encapsulated in a poly(ethylene glycol) hydrogel, Anal. Chem., 71: 3126-3132 (1999), and in U.S. Pat. No. 6,475,750. For example, a directly generated signal can be a signal that is produced when a conformational change occurs in a binding protein, such as when the protein binds specifically to its target

In one specific embodiment, the binding molecule is a protein that comprises a reporter group that is a luminescent label. The luminescent label may be a fluorescent label or a phosphorescent label. One particular embodiment of the present invention comprises the use of fluorescent labels, which may be excited to fluoresce by exposure to certain wavelengths of light.

In one specific embodiment, the reporter group attached to the binding protein is a fluorophore. As used herein, “fluorophore” refers to a molecule that absorbs energy and then emits light. Non-limiting examples of fluorophores useful as reporter groups in this invention include fluorescein, coumarins, rhodamines, 5-TMRIA (tetramethylrhodamine-5-iodoacetamide), Quantum Red™, Texas Red™, Cy3, N-((2-iodoacetoxy)ethyl)-N-methyl)amino-7-nitrobenzoxadiazole (IANBD), 6-acryloyl-2-dimethylaminonaphthalene (acrylodan), pyrene, Lucifer Yellow, Cy5, Dapoxyl® (2-bromoacetamidoethyl)sulfonamide, (N-(4,4-difluoro-1,3,5,7-tetramethyl- 4-bora-3a,4a-diaza-s-indacene- 2-yl)iodoacetamide (Bodipy507/545 IA), N-(4,4-difluoro-5,7-diphenyl- 4-bora-3a,4a-diaza-s-indacene- 3-propionyl)-N′-iodoacetylethylenediamine (BODIPY® 530/550 IA), 5-((((2-iodoacetyl)amino)ethyl)amino)naphthalene-1-sulfonic acid (1,5-IAEDANS), and carboxy-X-rhodamine, 5/6-iodoacetamide (XRIA 5,6). Many detectable intrinsic properties of a fluorophore reporter group may be monitored to detect glucose binding. Some of these properties which can exhibit changes upon glucose binding include fluorescence lifetime, fluorescence intensity, fluorescence anisotropy or polarization, and spectral shifts of fluorescence emission. Changes in these fluorophore properties may be induced from changes in the fluorophore environment such as those resulting from changes in protein conformation. Environment-sensitive dyes such as IANBD are particularly useful in this respect. Other changes of fluorophore properties may result from interactions with the analyte itself or from interactions with a second reporter group, for example when FRET (fluorescence resonance energy transfer) is used to monitor changes in distance between two fluorophores.

Fluorophores that operate at long excitation and emission wavelengths (for example, about 600 nm or greater excitation or emission wavelengths) are useful when the molecular sensor is to be used in vivo, for example, incorporated into an implantable biosensor device (the skin being opaque below 600 nm). Presently, there are few environmentally sensitive probes available in this region of the spectrum and perhaps none with thiol-reactive functional groups. However, thiol-reactive derivatives of Cy-5 can be prepared for example as taught by H. J. Gruber, et al, Bioconjugate Chem., (2000), 11, 161-166. Conjugates containing these fluorophores, for example, attached at various cysteine mutants constructed in binding proteins, can be screened to identify which results in the largest change in fluorescence upon analyte binding.

In one particular embodiment, the binding molecule is a mutant protein is a mutant GGBP comprising a luminescent label as the reporter group, The binding of glucose to this fluorescent-labeled GGBP should, in turn, alter the measured luminescence of the reporter group, and this change in the detectable characteristics may be due to an alteration in the environment of the label bound to the mutated GGBP.

It is also contemplated that other reporter groups, besides luminescent labels, may be used to provide the detectable signal. For example, electrochemical reporter groups could be used wherein an alteration in the environment of the reporter will give rise to a change in the redox state thereof. Such a change may be detected using an electrode. Furthermore, it is envisaged that other spectroscopically detectable labels, for example labels detectable by NMR (nuclear magnetic resonance), may be used.

The reporter group may be attached to the binding molecule by any conventional means known in the art. For example, if the binding molecule is a binding protein, the reporter group may be attached via amines or carboxyl residues on the protein. In particular, covalent coupling via thiol groups on cysteine residues may be exploited. For example, for mutated GGBP, cysteines located at position 11, position 14, position 19, position 43, position 74, position 107, position 110, position 112, position 113, position 137, position 149, position 152, position 213, position 216, position 238, position 287, and position 292 may be used.

Any thiol-reactive group known in the art may be used for attaching reporter groups such as fluorophores to an engineered or mutated protein's cysteine. For example, an iodoacetamide, bromoacetamide, or maleimide are well known thiol-reactive moieties that may be used for this purpose.

The compositions, devices and methods of the present invention also comprise a matrix that entraps the binding molecules. As used herein, “matrix” refers to essentially a three-dimensional environment which has at least one binding molecule immobilized therein for the purpose of measuring a detectable signal from ligand-protein interaction. The relationship between the constituents of the matrix and the binding molecule include, but are not limited to covalent, ionic, and Van der Wals interactions and combinations thereof. The spatial relationship between the matrix and binding molecules includes heterogeneous and homogeneous distribution within and or upon any or all of the matrix volume. The matrix may be comprised of organic, inorganic, glass, metal, plastic, or combinations thereof. The matrix may also allow the biosensor to be incorporated at the distal end of a fiber or other small minimally invasive probe to be inserted within the tissue of a patient, to enable an episodic, continuous, or programmed reading to the patient. Information from the biosensor to the patient may be provided, for example, by telemetry, visual, audio, or other means known in the art, for example, as taught in U.S. Pat. No. 5,517,313, U.S. Pat. No. 5,910,661, U.S. Pat. No. 5,894,351, and U.S. Pat. No. 5,342,789 as well as in Beach, R. D., et al. IEEE Transactions on Instrumentation and Measurement (1999) 48, 6, p.1239-1245. Information includes electrical, mechanical, and actinic radiation suitable for deriving analyte concentration or change in concentration, as is suitable.

As mentioned above, the binding molecule should be entrapped within a matrix, such as a hydrogel, which may then be used as an implantable device. As used herein, the term “entrap” and variations thereof is used interchangeably with “encapsulate” and is used to mean that the binding molecule is immobilized within or on the constituents of the matrix. The matrix can be in any desirable form or shape including one or more of disk, cylinder, patch, nanoparticle, microsphere, porous polymer, open cell foam, and combinations thereof, providing it permits permeability to analyte. The matrix additionally prevents leaching of the biosensor. The matrix permits light from optical sources or any other interrogating light to or from the reporter group to pass through the biosensor. When used in an in vivo application, the biosensor will be exposed to a substantially physiological range of analyte and determination or detection of a change in analyte concentration would be desired whereas the determination or detection includes continuous, programmed, and episodic detection means. Thus, in one embodiment of the present invention, the envisaged in vivo biosensor comprises at least one mutated binding protein in an analyte permeable entrapping or encapsulating matrix such that the mutated binding protein provides a detectable and reversible signal when the mutated binding protein is exposed to varying analyte concentrations, and the detectable and reversible signal can be related to the concentration of the analyte. The implantable biosensors may, in some embodiments, be implanted into or below the skin of a mammal's epidermal-dermal junction to interact with the interstitial fluid, tissue, or other biological fluids. Information from the implant to the patient may be provided, for example, by telemetry, visual, audio, or other means known in the art, as previously stated.

Preferably, the matrix is prepared from biocompatible materials or incorporates materials capable of minimizing adverse reactions with the body. Adverse reactions for implants include inflammation, protein fouling, tissue necrosis, immune response and leaching of toxic materials. Such materials or treatments are well known and practiced in the art, for example as taught by Quinn, C. P.; Pathak, C. P.; Heller, A.; Hubbell, J. A. Biomaterials 1995, 16(5), 389-396, and Quinn, C. A. P.; Connor, R. E.; Heller, A. Biomaterials 1997,18(24), 1665-1670.

In one aspect of the present invention, the binding molecule may be entrapped or encapsulated within a matrix that is derived substantially from a hydrogel. The term “hydrogel” is used to indicate a water-insoluble, water-containing material.

Numerous hydrogels may be used in the present invention. The hydrogels may be, for example, polysaccharides such as agarose, dextran, carrageenan, alginic acid, starch, cellulose, or derivatives of these such as, e.g., carboxymethyl derivatives, or a water-swellable organic polymer such as, e.g., polyvinyl alcohol, polyacrylic acid, polyacrylamide, polyethylene glycol, copolymers of styrene and maleic anhydride, copolymers of vinyl ether and maleic anhydride and derivates thereof. Derivatives providing for covalently crosslinked networks are preferred. Synthesis and biomedical and pharmaceutical applications of hydrogels based on, comprising polypeptides, have been described by a number of researchers. (See, e.g. “Biosensors Fundamentals and Applications”, edited by A. D. F. Turner, I. Karube and G. S. Wilson; published from Oxford University Press, in 1988). An exemplary hydrogel matrix derived from a water-soluble, UV crosslinkable polymer comprises poly(vinyl alcohol),N-methyl-4(4′-formylstyryl)pyridinium methosulphate acetal (CAS Reg. No. [107845-59-0]) available from PolyScience Warrington, Pa.

The polymers that are to be used in the matrices, such as hydrogels, used in the present invention may be functionalized. Of course, polymers used in other matrices may also be functionalized. That is, the polymers or monomers comprising the polymers should possess reactive groups such that the polymeric matrices, such as hydrogels, are amenable to chemical reactions, e.g., covalent attachment. As used herein and throughout, a “reactive group” is a chemical group that can chemically react with a second group. The reactive group of the polymer or monomers comprising the polymer may itself be an entire chemical entity or it may be a portion of an entire chemical entity, including, but not limited to single atoms or ions. Further, the second group with which the reactive group is capable of reacting can be the same or different from the reactive group of the polymer or monomers comprising the polymers. Examples of reactive groups include, but are not limited to, halogens, amines, amides, aldehydes, acrylates, vinyls, hydroxyls and carboxyls. In one embodiment, the polymers or monomers comprising the polymers of the hydrogel should be functionalized with carboxylic acid, sulfate, hydroxy or amine groups. In another embodiment of the present invention, the polymers or monomers comprising the polymers of the hydrogel are functionalized with one or more acrylate groups. In one particular embodiment, the acrylate functional groups are terminal groups. The reactive groups of the polymers or monomers comprising the polymers of the matrix may be reactive with any component of the matrix portion of the biosensor, such as, but not limited to, another polymer or monomer within the matrix, a binding protein and an additive.

Once formed, the core of any hydrogels used in the present invention should comprise polymers to form a polymeric hydrogel. Regardless of its application, the term “polymer” herein is used to refer to molecules composed of multiple monomer units. Suitable polymers which may be used in the present invention include, but are not limited to, one or more of the polymers selected from the group consisting of poly(vinyl alcohol), polyacrylamide, poly (N-vinyl pyrolidone), poly(ethylene oxide) (PEO), hydrolysed polyacrylonitrile, polyacrylic acid, polymethacrylic acid, poly(hydroxyethyl methacrylate), polyurethane polyethylene amine, poly(ethylene glycol) (PEG), cellulose, cellulose acetate, carboxy methyl cellulose, alginic acid, pectinic acid, hyaluronic acid, heparin, heparin sulfate, chitosan, carboxymethyl chitosan, chitin, collagen, pullulan, gellan, xanthan, carboxymethyl dextran, chondroitin sulfate, cationic guar, cationic starch as well as salts and esters thereof. The polymers of the matrix, such as a hydrogel, may also comprise polymers of two or more distinct monomers. Monomers used to create copolymers for use in the matrices include, but are not limited to acrylate, methacrylate, methacrylic acid, alkylacrylates, phenylacrylates, hydroxyalkylacrylates, hydroxyalkylmethacrylates, aminoalkylacrylates, aminoalkylmethacrylates, alkyl quaternary salts of aminoalkylacrylamides, alkyl quaternary salts of aminoalkylmethacrylamides, and combinations thereof. Polymer components of the matrix may, of course, include blends of other polymers. In one particular embodiment of the present invention, a hydrogel biosensor comprises a binding molecule and a matrix, with the matrix comprising a hydrogel of copolymers of (hydroxyethyl methacrylate) and methacrylic acid. In another particular embodiment of the present invention, a hydrogel biosensor comprises a binding molecule and a matrix hydrogel of copolymers of (hydroxyethyl methacrylate), methacrylic acid, and alkyl quaternary salts of methacrylamides.

The polymers used in the matrices can be modified to contain nucleophilic or electrophilic groups. Indeed, the polymers used in the present invention may further comprise polyfunctional small molecules that do not contain repeating monomer units but are polyfunctional, i.e., containing two or more nucleophilic or electrophilic functional groups. These polyfunctional groups may readily be incorporated into conventional polymers by multiple covalent bond-forming reactions. For example, PEG can be modified to contain one or more amino groups to provide a nucleophilic group. Examples of other polymers that contain one or more nucleophilic groups include, but are not limited to, polyamines such as ethylenediamine, tetramethylenediamine, pentamethylenediamine, hexamethylenediamine, bis-(2-hydroxyethyl)amine, bis-(2-aminoethyl)amine, and tris-(2-aminoethyl)amine. Examples of electrophilic groups include but are not limited to, succinimide esters, epoxides, hydroxybenzotriazole esters, oxycarbonylimidazoles, nitrophenyl carbonates, tresylates, mesylates, tosylates, carboxylates, and isocyanates. In one embodiment, the composition comprises a bis-amine-terminated poly(ethylene glycol).

The polymers should be capable of crosslinking, either physically or chemically, to form a matrix, such as a hydrogel. Physical crosslinking includes, but is not limited to, such non-chemical processes as radiation treatment such as electron beams, gamma rays, x-rays, ultraviolet light, anionic and cationic treatments. The crosslinking of the polymers may also comprise chemical crosslinking, such as covalent crosslinking. For example, a chemical crosslinking system may include, but is not limited to, the use of enzymes, which is well-known in the art. Another example of the chemical covalent crosslinking comprises the use of peroxide. Chemical crosslinking may occur when a crosslinking reagent reacts with at least two portions of a polymer to create a three-dimensional network. Covalent crosslinking may also occur when multifunctional monomers are used during the crosslinking process. For example, an acrylate monomer may be polymerized with a bifunctional acrylate monomer to form a crosslinked polymer. Any crosslinking reagent will be suitable for the present invention, provided the crosslinking reagent will at least partially dissolve in water or an organic solvent and can form the crosslinked polymer. For example, if the polymer is an amine-terminated PEG, the crosslinking reagent should be capable of reacting with the PEG-amine groups and be substantially soluble in water. In another example, (hydroxyethyl methacrylate) and methacrylic acid monomers can be polymerized with poly(ethylene glycol)-bis-alklyacrylate crosslinking agent in water or in dimethylformide to form polymeric hydrogels.

If the polymers to be crosslinked are functionalized with nucleophilic groups, such as amines (primary, secondary and tertiary), thiols, thioethers, esters, nitriles, and the like, the crosslinking reagent can be a molecule containing an electrophilic group. Examples of electrophilic groups have been described herein. Likewise, if polymers to be crosslinked are functionalized with electrophilic groups, the crosslinking reagent can be a molecule containing a nucleophilic group. It is understood that one skilled in the art can exchange the nucleophilic and electrophilic functional groups as described above without deviating from the scope of the present embodiment. It is also understood that the binding molecule can provide the requisite nucleophilic and electrophilic functional groups. For example, where the binding molecule is a protein, the nucleophilic and electrophilic functional groups may be present as naturally occurring amino acids in the protein, or may be introduced to the protein using chemical techniques described herein.

Other general methods for preparing or crosslinking polymers to form matrices such as hydrogels are well known in the art. For example, Ghandehari H., et al., J. Macromol. Chem. Phys. 197: 965 (1996); and Ishihara K, et al., Polymer J.,16: 625 (1984), all of which are hereby incorporated by reference, report the formation of hydrogels.

The binding molecules can be covalently attached to or non-covalently entrapped or encapsulated within a matrix, such as, but not limited to, a hydrogel. In one embodiment of the present invention, the binding molecules are covalently attached to, ie., entrapped within, a hydrogel. The covalent attachment of the binding molecule to the hydrogel should not interfere with the binding of the binding molecule to the target ligand. Furthermore, the covalent attachment of the binding molecule to the hydrogel should be resistant to degradation. The functional group in one embodiment, a polymer or other component of the hydrogel serves to couple the binding molecule to the hydrogel. The coupling of the binding molecule to the hydrogel can be accomplished in any number of ways. For example, coupling reactions between the hydrogel and binding molecule include, but are not limited to, diazonium coupling, isothiocyano coupling, hydrazide coupling, amide formation, disulfide coupling, maleic anhydride coupling, thiolactone coupling, and dichlotriazine coupling. These coupling reactions between two functional groups are well documented, and are considered well known to those skilled in the art. For example, an amino functional group in a binding molecule can be covalently coupled to a carboxyl functional group of one or more components of a hydrogel using coupling agents such as 1-ethyl-3-(3-dimethylaminopropyl)carbodiimide hydrochloride (EDC) or dicyclohexylcarbodiimide (DCC). It is understood that the amino and carboxyl functional groups of the binding molecule and one or more components of the hydrogel as described above can be transposed without deviating from the scope of the embodiment.

Other non-limiting examples of such coupling agents include, but are not limited to, benzyl carbamate and hydroxybenzotriazole, or bifunctional reagents such as N-succinimidyl 3-(2-pyridyldithio)propionate (SPDP), sulfo-LC-SPDP, succinimidyl 4-(N-maleimidomethyl)cyclohexane-1-carboxylate (SMCC), sulfo-SMCC, m-maleimidobenzoyl-N-hydroxysuccinimide ester (MBS), sulfo-MBS, N-succinimidyl (4-iodoacethyl)aminobenzoate (SIAB), sulfo-SIAB, succinimidyl 4-(p-maleimidophenyl)butyrate (SMPB), sulfo-SMPB, dithiobis (succinimidylpropionate), 3,3′-dithiobis (succinimidylpropionate), disuccinimidyl suberate, bis (sulfosuccinimidyl)suberate, disuccinimidyl tartarate (DST), sulfo-DST, bis[2-(succinimidooxycarbonyloxy)ethyl]sulfone (BSOCOES), sulfo-BSOCOES, ethylene glycolbis(disuccinimidylsuccinate (EGS), sulfo-EGS, etc. Other reagents that have several pendant functional groups such as thiol, hydroxyl, acyl chloride, sulfate, sulfonyl chloride, phosphate, phosphate chloride, and imide can also be conjugated to hydrogel using the above coupling agents and crosslinking agents.

The covalent attachment of a binding molecule to a the matrix, such as a hydrogel, can also be accomplished via photo polymerization and crosslinking either concurrently or subsequently to formation of the matrix. The photo polymerization and crosslinking of the polymer to binding molecule includes the use of photoinitiators that generate reactive species, such as free radicals or cationic centers, upon exposure to an energy source. Examples of photoinitiators that may be used include, but are not limited to, peroxides, ketones, and azo compounds. Specific examples of photoinitiators include, but are not limited to, 2-hydroxy-2-methylpropiophenone, benzoin, and 2,2-dimethoxy-2-phenyl-acetophenone, and the like. The energy from the energy source may be from anywhere in the electromagnetic spectrum, such as, but not limited to, radio waves, infrared light, visible light, ultraviolet light, X-rays and gamma rays. In one embodiment, the energy source used to polymerize and crosslink a binding protein to a hydrogel is ultraviolet light.

The covalent coupling of a binding molecule to the matrix, such as a hydrogel, can take place after hydrogel formation or during hydrogel formation. For example, the polymer and the binding molecule can be mixed with a crosslinking component, used in the formation of a hydrogel, in the presence of water to form the hydrogel biosensor of the present invention in a single or “one pot” process. The reaction can take place at room temperature or at an elevated temperature compatible with the binding molecule. The resultant binding activity of the hydrogel-attached binding molecule may be affected, for example, by binding molecule loading concentration, polymer concentration, and the molar ratio of nucleophilic groups to electrophilic groups, or cationic groups to either neutral or anionic groups. When the binding molecule is a binding protein, the protein may be randomly immobilized within the hydrogel. In one specific embodiment, amino groups from polymer components of a hydrogel are not only able to react with electrophilic groups to form the hydrogel, but they also prevent or inhibit excessive multiple site covalent attachment of the binding protein. Maintaining excess amino groups in the hydrogel components has been observed to maintain the binding protein activity of the biosensor. In another embodiment, the binding molecule or a functionally derivatized binding molecule may function as a monomeric component of, and co-polymerize with, other monomeric components of the hydrogel. For example, an acrylate functional group can be covalently attached to a binding molecule to provide a co-polymerizable component of a hydrogel.

Alternatively, the binding molecule can be covalently coupled to the matrix, such as a hydrogel, after matrix formation, creating a biosensor via a “two pot” process. In an exemplary embodiment, a hydrogel is first formed via the polymerization and crosslinking reaction, and unreacted monomers are washed from the hydrogel. The hydrogel is then placed in a buffer solution containing a binding molecule, and the solution is allowed to diffuse into the hydrogel. By way of example, a homobifunctional crosslinker comprising amine-reactive groups may be added to the buffer solution to couple a carboxyl functional group of the hydrogel with a carboxyl functional group of a binding molecule. The homobifunctional crosslinker can be the same or different from the crosslinker used when polymerizing and crosslinking the components that form the matrix.

Preparing the biosensor via the “two pot” method may also be accomplished by adding a crosslinker to the matrix, such as a hydrogel, after its formation, but before adding a binding molecule. The crosslinker is allowed to react with the matrix and the matrix is, in turn, contacted with a buffer solution containing the binding molecule. The coupling reaction can take place at a temperature compatible with the binding molecule. In addition, the amount of binding molecule attached to the matrices and the binding activity of the binding molecule may be controlled by pH. For example, a coupling reaction with a pH of about 7.0 should favor electrophilic groups reacting with the N-terminal amine of a protein, whereas a coupling reaction pH of about 9.0 should favor electrophilic groups reacting with lysine amine groups of a protein.

The binding molecule can also be covalently coupled to a preformed cross-linked matrix through site specific coupling. For example, when the binding molecule is a protein, site specific coupling to the hydrogel may be provided using free thiol groups at cysteine sites of the protein. An example of such a covalent coupling is described in U.S. application Ser. No. 10/428,295 filed May 2, 2003, which is hereby incorporated by reference. For site-specific attachment of a binding molecule, the matrix is first prepared with an excess of nucleophilic groups, such as amines and is then covalently coupled with a binding molecule using a heterobifunctional crosslinker such as, but not limited to sulfosuccinimidyl 6-[3′(2-pyridyldithio)-propionamide]hexanoate (sulfo-LC-SPDP). Sulfo-LC-SPDP reacts with amine and thiol groups, respectively, of the matrix and binding molecule. By this site-specific attachment, a binding protein can be covalently attached to the hydrogel while maintaining conformational freedom and analyte binding capability.

In one embodiment of the entrapment process, one or more hydrogels in water is added the mutated binding protein in an aqueous buffer solution having a pH in the range of about 4 to about 10 depending on the protein. Subsequent curing of the matrix, for example crosslinking, provides physical form. Using this technique and a conventional fabrication process (e.g. block casting, reverse emulsion polymerization, screen or contact printing, fluid-bed coating and dip or spin coating) one can obtain matrices in various configurations (e.g. granulates, nanoparticles, microparticles, monoliths, and thick and thin films) suitable for in vitro and in vivo use. In a specific embodiment, a thin film of the hydrogel biosensor can be prepared in sheet form or deposited on a sheet that is capable of being subsequently cut into strips for in vitro use.

In another embodiment of the present invention, binding proteins may be physically entrapped or encapsulated within the aforementioned matrices, such as, but not limited to, the aforementioned hydrogels. Such methods of physically entrapping binding molecules include one and two pot methods previously described herein, without the coupling reaction between the binding molecule and components of the matrix. In a specific embodiment, the physically entrapped or encapsulated binding protein hydrogel biosensor can be prepared in sheet form or deposited on a sheet that is capable of being subsequently cut into strips for in vitro use.

In one embodiment of the present invention, the matrix, such as a hydrogel, may further comprise one or more additives. For example, one or more additives that may be included in the matrix include, but are not limited to, carbohydrates such as monosaccharides, disaccharides, polysaccharides, amino acids, oligopeptides, polypeptides, proteoglycans, glycoprotein, nucleic acids, oligonucleotides, lipids, fatty acids, natural or synthetic polymers, small molecular weight compounds such as antibiotics, drugs or drug candidates, and derivatives thereof. In one particular embodiment, the hydrogel biosensors further comprise at least one carbohydrate or alcohol derivative thereof. More particularly, the hydrogel biosensor includes at least one compound selected from the group consisting of allose, altrose, glucose, mannose, gulose, idose, galactose, talose, ribulose, fructose, sorbose, tagatose, sucrose, lactose, maltose, isomaltose, cellobiose, trehalose, mannitol, sorbitol, xylitol, maltitol, dextrose, and lactitol. Such additives can provide enhanced storage stability of a binding molecule hydrogel biosensor. In a specific embodiment, a trehalose additive is added to a binding protein hydrogel biosensor to provide improved lyophilized storage stability described herein.

The matrix may, in one embodiment, be comprised of modified sol-gels. Modified sol-gels includes at least partial cured (or gelled) preparations comprised of permeable metal oxide glass structures containing in addition to the sol-gel precursor materials, preferably one or more organic components which hydrolytically condense along with the sol-gel precursor such that the resultant sol-gel matrix imparts properties suitable for, by example, implantation. Suitable properties include low volume shrinkage over time, resistance to cracking and other physical defects, maintenance of protein function, and compatibility with the protein and or reporter group, and compatibility with the animal or subject to which it may be implanted. Suitable organic materials include polyols such as glycerol, ethylene glycol, propylene glycol, polyethylene glycol, and the like for example, as taught by Gill and Ballesteros Journal of the American Chemical Society 1998, 120(34), 8587-8598. It is understood that those skilled in the art can appreciate the attributes described are generally not predictable for a given protein/sol-gel/reporter group combination, thus optimization of sol-gel precursor, organic component and protein solution materials may be expected for any given binding protein-reporter pair. It has been found by the applicants that such optimization may provide for unexpected enhanced signal, shifted binding constants, improved physical performance attributes of the matrix, and combinations thereof relative to that of other matrices or aqueous solutions thereof. Optimization of performance attributes of the binding molecule-reporter pair and functional performance attributes of the matrix encapsulating the binding molecule may be achieved, for example, by way of combinatorial methods or other statistical based design methods known in the art.

Sol-gel matrices useful for the present invention include material prepared by conventional, well-known sol-gel methods and include inorganic material, organic material or mixed organic/inorganic material. The materials used to produce the sol-gel can include, but are not limited to, aluminates, aluminosilicates and titanates. These materials may be augmented with the organically modified silicates (Ormosils) and functionalized siloxanes, to provide an avenue for imparting and manipulating hydrophilicity and hydrophobicity, ionic charge, covalent attachment of protein, and the like. As used herein the term “hydrolytically condensable siloxane” refers to sol-gel precursors having a total of four substituents, at least one, preferably two, and most preferably three or four of the substituents being alkoxy substituents covalently bound to silicone through oxygen and mixtures thereof. In the case of three, two, and one alkoxy substituent precursors, at least one of the remaining substituents preferably is covalently bound to silicone through carbon, and whereas the remaining substituent contains organic functionality from alkyl, aryl, amine, amide, thiol, cyano, carboxyl, ester, olefinic, epoxy, silyl, nitro, and halogen.

In one embodiment of the encapsulation process, one or more of hydrolytically condensable siloxane is hydrolyzed in water, either spontaneously or under acid or base catalysis to form derivatives with an organic polyol component present in a molar amount relative to the hydrolytically condensable siloxane up to about 10:1 to 1:10, preferably to about 5:1 to 1:5, and most preferably to about 1:1. To this mixture, prior to final gellation, is added the mutated binding protein in an aqueous buffer solution having a pH in the range of about 4 to about 10 depending on the protein. At least partial condensation reactions give rise to the final matrices.

In another embodiment, the hydrolytically condensable siloxane hydrolyzed in water, either spontaneously or under acid or base catalysis to form derivatives with the organic polyol, is mixed with a water soluble polymer component. Suitable water soluble polymers include polyvinyl alcohol (PVA), poly-(maleic acid co-olefin) sodium salt (PMSA), poly-(vinylsulfonic acid) sodium salt (PVSA), and polyvinyl pyrollidone (PVP). Poly-(maleic acid co-olefin) includes copolymers of maleic anhydride with styrene, vinyl ether, and C1-C8 olefins and salts thereof, for example, sodium, potassium, ammonium, tetraakylammonium, and the like. Preferably, the water soluble polymer component is from 0 to about 30% by weight of the sol-gel composition.

In another embodiment the hydrolytically condensable siloxane hydrolyzed in water, either spontaneously or under acid or base catalysis to form derivatives with the organic polyol, is mixed with one or more functionalized silicone additives (FSA) in amounts from 0 to about 0.6% mole ratios to hydrolytically condensable siloxane. Exemplary FSA's include alkyl derivatives: for example, methyltrimethoxysilane (MTMOS): amine derivatives: for example, 3-aminopropyl triethoxysilane (ATEOS); and bis silane derivatives: for example, (bis(3-methyldimethoxysilil)propyl)polypropylene oxide (BIS).

In another embodiment, both the water soluble polymer component and the functionalized silicone additive are mixed together with the hydrolytically condensable siloxane hydrolyzed in water, either spontaneously or under acid or base catalysis to form derivatives with the organic polyol, to provide for a matrix suitable for entrapment or encapsulation of the binding protein. Using the afore-mentioned sol-gel technique and a conventional fabrication process (e.g. block casting, reverse emulsion polymerization, screen or contact printing, fluid-bed coating and dip or spin coating) one can obtain aerogel- or xerogel-matrices in various configurations (e.g. granulates, nanoparticles, microparticles, monoliths, and thick and thin films) suitable for use in vitro and in vivo.

In another embodiment the matrix, such as a hydrogel, may be used in combination with dialysis membranes. The dialysis membranes can be constructed to physically encapsulate or entrap the hydrogel matrix containing the binding molecule. Covalent attachment of the matrix and/or the binding molecule to the dialysis membrane is considered within the scope of the as described embodiment. The membrane should be chosen based on its molecular weight cut-off such that analytes of interest can readily permeate the membrane whilst high molecular weight materials would be restricted from entering, or in the case of the mutated binding proteins, leaving the membrane matrix. The molecular weight cut-off required would be such as to meet the afore-mentioned requirement and is within the skill of one familiar with this art. Typically, membranes having molecular weight cut-off between about 1000 to about 25,000 Daltons are suitable. Using this technique, matrices in various configurations and shapes suitable for use in vitro and in vivo can be prepared.

It is also contemplated that matrices containing the binding protein and reporter group be combinations of one or more hydrogel, sol-gel, and dialysis membranes. For example, a protein entrapped or encapsulated within a hydrogel or sol gel can be placed within a dialysis membrane of a suitable shape and size as would provide for implantation within a subject, or to manipulate mass-transport properties or permeablity of the analytes with respect to the matrix.

The matrix entrapped or encapsulated binding molecule biosensors of this invention are capable of measuring or detecting micromolar (10⁻⁶ molar) to molar analyte concentrations without reagent consumption. In some embodiments, their sensitivity to analyte may enable the biosensors to be used to measure the low analyte concentrations known to be present in low volume samples of interstitial fluid. The implantable biosensors may, in some embodiments, be implanted into or below the skin of a mammal's epidermal-dermal junction to interact with the interstitial fluid, tissue, or other biological fluids. In a specific embodiment, a binding protein biosensor of the present invention may provide for the means to monitor analyte continuously, episodically, or “on-demand” as would be appropriate to the user or to the treatment of a condition.

The present invention also relates to methods of detecting the presence of an analyte (ligand) in a sample using the biosensors of the present invention. As used herein, the terms “ligand” and “analyte” are used interchangeably and are used to indicate the molecule to which the binding molecule of the biosensors will specifically bind. The analyte or ligand measured in the methods described herein is not labeled with a reporter group. As used herein, a sample can be any environment that may be suspected of containing the analyte to be measured. Thus, a sample includes, but is not limited to, a solution, a cell, a body fluid, a tissue or portion thereof, and an organ or portion thereof. When a sample includes a cell, the cell can be a prokaryotic or eukaryotic cell, for example, an animal cell. Examples of animal cells include, but are not limited to, insect, avian, and mammalian such as, for example, bovine, equine, porcine, canine, feline, human, and nonhuman primates. The scope of the invention should not be limited by the cell type assayed. Examples of biological fluids to be assayed include, but are not limited to, blood, urine, saliva, synovial fluid, interstitial fluid, cerebrospinal fluid, lymphatic fluids, semen, ocular fluid, bile and amniotic fluid. The scope of the methods of the present invention should not be limited by the type of body fluid assayed. The terms “subject” and “patient” are used interchangeably herein and are used to mean an animal, particularly a mammal, more particularly a human or nonhuman primate.

In one embodiment, for measuring the concentrations of a target analyte, the biosensors of the present invention may be contacted with analyte-free solutions (control), such as buffers, and the directly generated signal measured. The value of the fluorescence measured may be, but is not limited to, intensity, rate-based or lifetime. The fluorescent measurement can, in turn, be directly or indirectly tied to the concentration of measured analyte. For example, the biosensors can be contacted with a sample suspected of containing an analyte to be measured, and the intensity of the directly generated signal is measured at least once. The sequence in measuring the intensity of the control and experimental signals is not important and can be performed in any order. Any differences in the generated signals are an indication of the presence or absence of the analyte in the sample or control. Furthermore, measurements of the generated signal can be taken either continuously, episodically, or sequentially to monitor changes in the concentration of the analyte in the sample. Once the control or baseline signal is established, the subsequently measured signals can be measured continuously or at discrete times.

The comparison of the signals can be qualitative or quantitative. Furthermore, the quantitative differences can be relative or absolute. Of course, the differences in signal may be equal to zero, indicating the absence of the analyte sought. The quantity may simply be the measured signal without any additional measurements or manipulations. Alternatively, the difference in signals may be manipulated mathematically or in an algorithm, with the algorithm designed to correlate the measured signal value to the quantity of analyte in the sample. The quantity may be expressed as a difference, percentage or ratio of the measured value of the analyte to a measured value of another compound including, but not limited to, a standard. The difference may be negative, indicating a decrease in the amount of measured analyte. The quantity may also be expressed as a difference or ratio of the analyte to itself, measured at a different point in time.

### EXAMPLES

The following examples illustrate certain preferred embodiments of the instant invention, but are not intended to be illustrative of all embodiments. Labeled mutated maltose binding protein S337C MBP with fluorophore reporter probe NBD used herein in accordance with the procedure set forth by Gilardi, A. et al. (Anal. Chem. 1994, 66, 3840-3847). Fluorescence emission spectra of mutated, labeled protein was measured using an SLM Aminco fluorimeter (Ontario, Canada) with slit settings of 8 and 4 for excitation and settings of 5 and 5 on the MC250 emission monochromator to compare the ligand-binding performance of the entrapped fluorophore-labeled proteins in various matrices to the performance of the same proteins in solution. The initial fluorescence emission intensity is defined as Iₒ. The relative ratio of the emission intensity maxima in the presence of the protein's respective ligand (I_{f}) to the ligand's absence (Iₒ) is defined as ΔF. It is understood that such a dimensionless value may also be expressed as a ratio of signal in the presence of ligand to a fixed or known quantity of ligand instead of an absence of ligand. In addition, a value defined as Qf, the ratio of fluorescence at a saturated or infinite ligand concentration (F_{inf}) and fluorescence at zero ligand concentration (F₀), may be used. Saturated or infinite ligand concentration may be approximated using a ligand concentration above the equilibrium dissociation constant of the binding molecule. The terms ΔF and Qf are used interchangeably herein and are intended to represent essentially the same dimensionless value.

Binding constants were determined by titration of increasing concentrations of glucose into a protein solution with mixing following each addition of glucose. Slit settings were the same as listed above. The Kd was determined from the following relationships as adapted from Pisarchick and Thompson (1990):

$$F = {F}_{inf} + \frac{{F}_{0} - {F}_{inf}}{1 + x / Kd} \qquad ( 1 )$$

where F is fluorescence intensity, F_{inf} is fluorescence at infinity, F₀ is fluorescence at zero glucose, and x is the free concentration of glucose ([Glc]_{free}) as determined by the relationship:

$${[ GLc ]}_{free} = \frac{\begin{matrix}{[ GLC ]}_{tot} - {[ Prot ]}_{tot} - Kd + \\ \sqrt{( {[ Glc ]}_{tot} - {[ Prot ]}_{tot} - Kd ) 2 + 4 * {[ Glc ]}_{tot}} * Kd\end{matrix}}{2}$$

where [Glc]ₜₒₜ and [Prot]ₜₒₜ are the total concentrations of glucose and protein, respectively.

### Example 1

### Expression of Mutant GGBP without Histidine Tags

This example describes the method for the expression and purification of mutant Proteins Without Histidine Tags. GGBP is coded by the Mg1B-1 gene in E. coli. This protein was altered by introducing the amino acid cysteine at various positions through site-directed mutagenesis of the Mg1B-1 gene. These proteins were then expressed in E. coli and purified.

Cassette mutagenesis of Mg1B-1 was accomplished as follows. The wild-type Mg1B-1 gene was cloned into a pTZ18R vector (Dr. Anthony Cass, Imperial College, London, England). Mutant plasmids were generated from this parent plasmid using cassette mutagenesis producing randomized amino acid sequences, essentially as described by Kunkel (1991) and cloned in E. coli JM 109 (Promega Life Science, Madison, Wis.). Mutant plasmids were identified by sequencing. The mutant protein was induced in JM109 and purified as described below. An E. coli JM109 colony containing the mutant plasmid was grown overnight at 37° C. with shaking (220 rpm) in LB broth containing 50 μg/mL ampicillin (LB/Amp). The overnight growth was diluted 1:100 in 1 L fresh LB/Amp and was incubated at 37° C. with shaking until the OD₆₀₀ of the culture was 0.3-0.5. Expression of the mutant was induced by the addition of 1 mM IPTG (Life Technologies, Gaithersburg, Md.) final concentration with continued incubation and shaking at 37° C. for 4-6 hours. The cells were harvested by centrifugation (10,000×g, 10 min, 4° C.).

The mutant protein was harvested by osmotic shock and was purified by column chromatography. The cell pellet was resuspended in a sucrose buffer (30 mM Tris-HCL pH 8.0, 20% sucrose, 1 mM EDTA), incubated at room temperature for 10 min, and then centrifuged (4000×g, 15 min, 4° C.). The supernatant was poured off and kept on ice. The cell pellet was resuspended, and 10 mL ice cold, sterile deionized H₂O was repeated, and the suspension was incubated on ice and centrifuged. The remaining supernatant was pooled with the other collected supernatants and was centrifuged once again (12,000×g, 10 min, 4° C.). The pooled shockate was filtered through a 0.8 μm and then a 0.45 μm filter. Streptomycin sulfate (Sigma Chemical Co., St. Louis, Mo.), 5% w/v, was added to the shockate and was stirred once for 30 min followed by centrifugation (12,000×g, 10 min, 4° C.). The shockate was then concentrated using the Amicon Centriprep 10 (10,000 MWCO) filters (Charlotte, N.C.) and dialyzed overnight against 5 mM Tris-HCl pH 8.0, 1 mM MgCl₂. The dialyzed shockate was centrifuged (12,000×g, 30 min, 4° C.). The resulting supernatant was added to a pre-equilibrated DEAE Fast Flow Sepharose column (Amersham Pharmacia Biotech, Piscataway, N.J.) at 0.5 mL/min. The column was washed with 5-10 column volumes. A linear gradient from 0-0.2 M NaCl was applied to the column and fractions were collected. The mutant protein containing fractions were identified by SDS-PAGE with Coomassie Brilliant Blue staining (mw. Approx. 32 kDa). The fractions were pooled and dialyzed overnight (4° C.) against phosphate buffered saline (PBS) or 10 mM ammonium bicarbonate (pH 7.4) concentrated using Amicon Centriprep 10 filters, and stored at 4° C. or −20° C. with glycerol. The ammonium bicarbonate dialyzed protein was lyophilized.

### Example 2

### Expression of Mutant GGBP with Histidine Tags

This example describes the expression and purification of mutant GGBPs containing Histidine Tags. GGBP mutants were engineered by either site-directed mutagenesis or the cassette mutagenesis. Site-directed mutagenesis (QuikChange, Stratagene, La Jolla, Calif.) was performed to alter individual amino acids in the pQE70 vector by replacing one amino acid with another, specifically chosen amino acid. The cassette mutagenesis method (Kunkel 1991) was performed to randomize amino acids in a specified region of the GGBP gene. The mutated cassettes were then subcloned into the pQE70 expression vector. The pGGBP-His plasmid contained the GGBP gene cloned into the pQE70 expression vector (Qiagen, Valencia, Calif.). This construct places six histidine residues on the C-terminus of the GGBP gene. E. coli strain SG13009 was used to over express mutant GGBP-His following standard procedures (Qiagen). After over expression of a 250 mL culture, the cells were collected by centrifugation (6000 rpm) and resuspended in 25 mL bugbuster (Novagen, Madison, Wis.). Lysozyme (25 mg was added to the lysate and the mixture was gently mixed at room temperature (RT) for 30 min. Clear lysate was produced by centrifugation (6000 rpm) and to this, 0.5 ml imidizole (1 M) and 3 ml of Ni-NTA beads (Qiagen) was added. After 30 minutes of gently mixing at RT, the mixture was centrifuged (6000 rpm) and the lysate removed. The beads were washed with 25 ml of solution (1M NaCl, 10 mM tris, pH 8.0) and recentrifuged. The mutant GGBP-His was eluted from the beads by adding 5 mL solution (160 mM imidazole, 1 M NaCl, 10 mM Tris, pH 8.0) and mixing for 15 min. The protein solution was immediately filtered through a Centriplus YM-100 filter (Amicon, Charlotte, N.C.) and then concentrated to 1-3 mg/ml using a Centriplus YM-10 filter. The protein was dialyzed overnight against 2 L of storage solution (1 M NaCl, 10 mM Tris, 50 mM NaPO₄, pH 8.0).

### Example 3

### Labeling of a Binding Protein with a Reporter Group

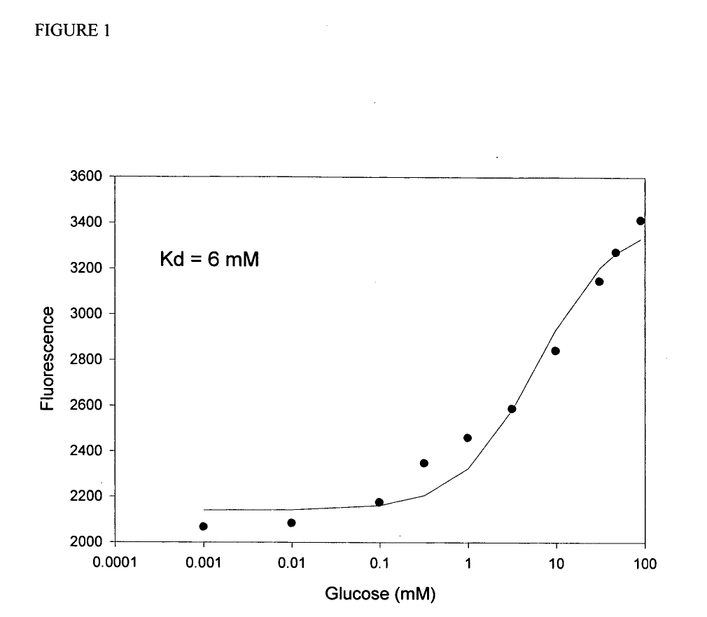

This example describes generically the labeling of binding protein with reporter probe. An aliquot of mutant GGBP containing cysteine (4.0 nmol) in PBS was treated with 2 mM dithiothreitol (5 μL, 10 nmol) for 30 min. A stock solution of N,N′-dimethyl-N-(iodoacetyl)-N′-(7-nitrobenz-2-oxa-1,3-diazol-4-yl)ethylenediamine (IANBD amide, 0.5 mg) was prepared in DMSO (100 μL, 11.9 mM) and 3.36 μL (40 nmol) was added to the protein. The reaction proceeded at room temperature for 4 h on a Dynal rotamix in the dark. The labeled protein was purified by gel filtration on a NAP-5 column (Amersham Pharmacia). The labeling rations were determined using an estimated extinction coefficient (50 mM⁻¹ cm⁻¹) for GGBP that was calculated in GeneWorks 2.45 (IntelliGenetics), ε₄₇₈ (IANBD amide)=25 mM⁻¹cm⁻¹), and a measurement of O.D. for a standard solution of IANBD amide at 280 nm and 478 nm. The dye concentration in the protein was calculated as C_{dye}=ε_{478/}A₄₇₈. The absorbance of protein at 280 nm was calculated as Aₚᵣₒₜ₍₂₈₀₎=Aₜₒₜₐₗ₍₂₈₀₎−A_{dye(280)}, where A_{dye(280)}=A₄₇₈×(A₂₈₀/A₄₇₈)standard. The concentration of protein was then Cₚᵣₒₜ₍₂₈₀₎=ε₂₈₀/Aₚᵣₒₜ₍₂₈₀₎. FIG. 1 illustrates the change in fluorescence response to glucose concentration of a representative example, A213C/L238C NBD amide GGBP H₆ in solution. Table 1 summarizes the change in fluorescence of various GGBP mutants labeled with reporter groups, including reporting groups having either excitation or emission maximum of at least 600 nanometers. Table 2 summarizes the change in fluorescence, and determined Kd values of mutations of one, two, three, and four amino acid substitutions. This data clearly shows mutations of the GGBP labeled with a reporter group can provide desirable attributes as glucose biosensors. The data shows the mutation-reporter group relationship for the samples tested.

TABLE 1
Percent Change in Fluorescence for GGBP Mutants¹

| | Excitation/ | | | | A213C | |
|---|---|---|---|---|---|---|
| Dye | emission (nm) | S112C | M182C | A213C | His₆ | M216C |
| IANBD amide | 470/550 | 0 | 4 | 3 | 51 | 7 |
| IANBD ester | 470/550 | | | | | |
| IAEDANS | 336/490 | −7 | −8 | 0 | | −9 |
| Bodipy530/550 IA | 530/550 | 7 | −10 | 33 | | 4 |
| XRIA 5, 6 | 575/600 | −21 | −19 | −38 | | −15 |
| Lucifer Yellow IA | 426/530 | | | −14 | | −3 |
| Bodipy 507/545 IA | 507/545 | | | 25 | | −3 |
| Cy5 | 640/660 | 2 | 0 | 11 | | −7 |
| Texas Red-maleimide | 580/610 | | | | −13 | |
| Dapoxyl | 375/580 | 15 | 7 | 12 | | 2 |

¹From 0 to 1 mM glucose at 0.5 μM [dye]. Unless otherwise indicated all mutants were without histidine tags.

TABLE 2
Summary of GGBP-H6 NBD Mutations

| | Solution | | | |
|---|---|---|---|---|
| Identification | F(%)¹ | Kd(mM)² | Dye/Prot | Seqncd |
| wild type | intrinsic | 0.0002 | — | for/rev |
| A1C | | — | — | — |
| K11C | 10 | — | 1.8 | rev |
| D14C | 1 | — | 1.5 | rev |
| V19C | −56 | 0.0001 | 0.38 | — |
| N43C | 40 | 0.0002 | 0.28 | — |
| G74C | −3 | 0.0009 | 1.43 | — |
| Y107C | −30 | 0.001 | 0.93 | for |
| T110C | −9 | | — | for/rev |
| S112C | 220 | 0.05 | 1.15 | — |
| S112C, L238S | 6 | — | 1.5 | — |
| K113C | 15 | — | 0.65 | — |
| K137C | −5 | 0.00004 | 1.17 | — |
| E149C | 300 | 0.0002 | 0.96 | — |
| E149C, A213R | 660 | 1 | 1.1 | for/rev |
| E149C, K223N | — | — | — | — |
| E149C, L238S | 660⁴ | 0.08 | 1.36 | for/rev |
| E149C, N256S | 1 | — | 0.93 | for/rev |
| E149C, M182C, | 200 | 216⁶ | 3.2 | for/rev |
| A213C, L238S | | | | |
| E149C, A213S, L238S | 480 | 0.47 | 0.76 | for/rev |
| E149C, A213R, L238S | 500 | 35 | — | — |
| H152C, A213R | −3 | — | 1.2 | for/rev |
| H152C, A213S | 100 | 0.16 | — | — |
| H152C, K223N | 200 | 0.003 | 1 | for |
| M182C | 11 | — | — | for/rev |
| A213C | 50 | 0.124 | 0.68 | for/rev |
| A213C, L238C | 24, 67³ | 6 | 1.4 | for/rev |
| M216C | 67 | 0.008 | 0.91 | for |
| L238C | −6, +3³ | — | 1.3 | for/rev |
| D287C | 4 | — | 1.1 | for |
| R292C | −34 | 0.0008 | 1.5 | for |

¹% Fluorescence intensity change from 0 to 1 mM Glc at 0.5 μM [NBD]
²Kd measured at 0.1 μM [dye]
³% Fluorescence change when measured from 0 to 100 mM Glc
⁴% Fluorescence change when measured from 0 to 10 mM Glc
⁵Estimated; Sigma Plot calc. did not converge
⁶Estimated; curve did not reach saturation
Seqncd = sequencing,
for = forward;
rev = reversed;
for/rev = both

### Example 4

### Immobilization of a Binding Protein

This example describes the immobilization of a biosensor of the instant invention using glycerol modified silicate condensate (GMSC). The additions of glycerol directly followed the initial tetraethoxyorthosilicate (TEOS) or tetramethoxyorthosilicate (TMOS) acid hydrolysis. A range of hydrolysis times, pH levels, reagent addition order, and TEOS:glycerol ratios were evaluated to determine the optimal conditions for beginning the glyceration reaction. Preferred conditions were found using an interval of 10 to 30 minutes between hydrolysis and glycerol addition, a pH range of between 0.5 and 1, and a 1:1 mole ratio of TEOS to glycerol. The following describes a modified procedure of Gill and Ballesteros for a TEOS-based glycerol modified silicate condensate (GMSC) preparation using the following ratios of reagents: TEOS or TMOS:1; H2O:1, Methanol:4, Glycerol:1. TEOS or TMOS in methanol was added to a flask and cooled to 0° C. over ice. Next 0.6M HCl was added drop-wise to the solution. After 20 minutes of stirring, glycerol was added drop-wise. The reaction was warmed slowly over 1-2 hours to 20-25° C. Following this the reaction vessel was heated further and maintained at a temperature range of 60-70° C. under nitrogen for between 36 and 42 hours. The optimal time was 40 hours. Incomplete glyceration was indicated by an observable phase separation for reactions stopped before 36 hours. Reactions maintained beyond 42 hours produced GMSC sol-gel monoliths with greatly reduced physical properties, for example, increased brittleness. Following the 40 hour reaction at 60-70° C., the solution volume was reduced by rotary evaporation until it was viscous and transparent, at which point methanol was added to the solution in a 4:1 ratio by weight. This GMSC solution proved to be stable and provided consistent results for several months when stored at freezer temperature. When the GMSC solution was to be used, methanol was removed by rotary evaporation and distilled water was added in a 1:1 ratio by weight to the GMSC reagent to catalyze the final hydroylsis/gelation. Monoliths, thin films, and powders were created with this procedure using an appropriate container to function as a mold. The GMSC sol-gel monoliths were not brittle and had shrinkage of about 8% after curing at 4° C. at 50% relative humidity for 2 weeks (% shrinkage was the average of changes in diameter and length measured with a microcaliper and compared to original mold dimensions). Electron microscopy (SEM) further illustrated the significant improvements in surface fracturing between monoliths created with TEOS hydrolysis and the monoliths created through the GMSC procedure described above. This set of experiments demonstrates how sol-gels with improved physical characteristics can be produced in accordance with the methods taught in the instant invention.

### Example 5

### Optimization of Sol-Gels

This example describes further optimization of physical properties by GMSC sol-gels in which glycerol has been partly substituted with either ethylene glycol (EG) or polyethylene glycol (PEG). Ethylene glycol (EG) was evaluated as a substitute for glycerol in mixtures where the ratio of glycerol and EG was varied but the mole ratio of total glycerol and EG was maintained constant relative to other reagents. Sol-gel monoliths were prepared by the procedure described in the preceding example, cured for two weeks at 4° C. and 50% relative humidity and their % shrinkage was determined and stated in Table 3. % Shrinkage is defined as the average of the decrease in length and diameter versus original dimensions. Monoliths used for determination of shrinkage had no protein/fluorophore present. For fluorescence measurements, the samples listed in Table 1 were prepared containing H152 GGBP-H6 NBD (from Example 3) as will be described shortly.

TABLE 3
Average % shrinkage and ΔF of sol-gel Matrix after 2 weeks.

| | | ΔF |
|---|---|---|
| | Average % | (10 mM |
| Sol-gel Matrix | shrinkage | Glucose) |
| 1. Solution (H152 GGBP-H6 NBD | Not Applicable | 1.53 |
| 0.8-1.2 μM) | | |
| 2. TEOS | 35.95 +/− 0.24 | 1.39 |
| 3. GMSC-TEOS | 8.01 +/− 0.19 | 1.57 |
| 4. GMSC-TEOS 15 wt % PMSA, | 3.99 +/− 0.27 | — |
| 0.145 mol % MTMOS | | |
| 5. 1% EG/GMSC-TEOS | 3.10 +/− 0.17 | 1.53 |
| 6. 5% EG/GMSC-TEOS | 2.48 +/− 0.15 | 1.47 |
| 7. 10% EG/GMSC-TEOS | — | 1.37 |
| 8. 20% EG/GMSC-TEOS | — | 1.34 |

The 1% and 5% EG/GMSC sol-gels (entries 5 and 6 respectively in Table 3) were found to have significantly less % shrinkage than either the plain TEOS sol-gels or GMSC modified TEOS sol-gels (entries 2 and 3 respectively in above Table 3). Polyethylene Glycol (PEG) was also evaluated qualitatively as a partial substitute for glycerol in similar proportions in GMSC sol-gels and produced monoliths with favorable surface properties and rubber-like flexibility. In summary, partial substitution of either ethylene glycol (EG) or polyethylene glycol (PEG) for glycerol in GMSC sol-gels provides improvements in physical properties, for example, minimized shrinkage and reduced surface fracturing. These sol-gel matrices containing binding protein were found to possess performance equal to or better than that of protein in solution.

### Example 6

### Entrapment of Binding Proteins in Sol-Gel

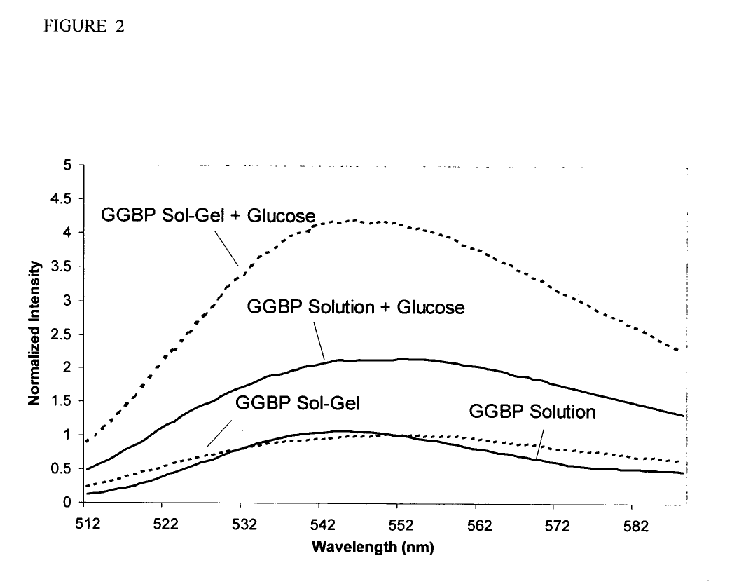

This example describes the addition of polymer and organic polyol additives to optimize the GMSC sol-gels for entrapping binding proteins to both maintain and enhance their spectral properties upon ligand binding. The binding proteins were labeled with a fluorophore (as described in example 3). The protein solutions were added during the final hydrolysis/gelling step described previously to produce final concentrations of 2-4 μM protein within the sol-gel. The polymer additives and FSA's were obtained from Sigma-Aldrich Chemicals (St. Louis, Mo.). Polymer additives were evaluated in amounts between 0 to about 30 wt. Functionalized silicone additives (FSA) were evaluated as additives to the GMSC sol-gels in amounts from 0 to about 0.6% mole ratio. Thus, rotary evaporation of the GMSC reagent to remove methanol from its storage solution was followed by reconstitution in water in a 1:1 ratio by weight. To a 400 μL aliquot of this mixture, 800 μL of buffer (HEPES, PBS or Tris) with a premixed water soluble polymer additive was added along with any FSA-modified GMSC. A mutated binding protein in solution was then, and after thorough mixing, 100 μL of the mixture was dispensed into a 96 well microplate (Falcon white flat bottom plates, product # 35-3941, BD Labware, N.J.). The sol-gel containing microplates were cured 12-18 hours at 4° C. and 50% relative humidity. GMSC-BIS was prepared by the same procedure as the TEOS-based GMSC, but with substitution of (Bis(3-methyldimethoxysilyl)propyl) polypropylene oxide for TEOS. GMSC-MTMOS and GMSC-ATEOS were prepared similarly except that the hydrolysis was carried out with either 10% of the amount of acid, or no acid in the hydrolysis step, respectively compared to the TEOS-based GMSC procedure. Fluorescence emission was measured with a Varian Cary Eclipse scanning fluorometer with microwell plate adapter (Varian Instruments, Victoria, Australia). Excitation was at 475 nm and emission recorded from 500 to 600 nm, typically monitoring emission maximum peak fluorescence. Slit widths were 5 nm for excitation and 10 nm for emission. Individual Iₒ determinations were made for each well and 100 μL of a ligand solution (1 mM maltose in the case of S337C MBP, and 10 mM glucose in the case of Hi152C GGBP, or 100 mM glucose in the case of A213C/L238C) was added and I_{f} readings were obtained, from which ΔF values were calculated. The modified sol-gel entrapped proteins exhibited greater initial fluorescence (Iₒ) in the absence of ligand when compared to equivalent concentrations of the same protein in solution. FIG. 2 shows the fluorescence emission before and after glucose addition for GGBP H152 His6 NBD in the H152 optimized sol-gel and in solution. The Iₒ spectra for each experiment were normalized to a maxima of 1.0. The figure shows about 2-3-fold enhancement of ΔF obtained for the optimized sol-gel matrices containing binding protein when exposed to analyte in comparison to protein in solution. Thus, after optimization of the sol-gel formulations for each protein, an enhancement of ΔF was observed. It should be noted that emission maximum may be shifted for sol-gel entrapped protein-reporter group samples as compared to solution. In addition, these modified sol-gel matrices provide improved physical properties as shown in entry 4 of Table 3. Table 4 shows an approximate range of components of formulations giving improved response for each of the individual proteins evaluated.

TABLE 4
Optimized sol-gel formulations for H152C GGBP His6-NBD, A213C/L238C
GGBP His6-NBD, and S337C-MBP-NBD. (GGBP = glucose/galactose
binding protein; MBP = maltose binding protein; NBD = N-(acetoxy)ethyl)-
N-methyl)amino-7-nitrobenzoxadiazole.

| | | |
|---|---|---|
| H 152 C GGBP-NBD | A213C/L238C GGBP-NBD | S337C MBP-NBD |

| | | |
|---|---|---|
| Range | Range | Range |

| | | | | | | |
|---|---|---|---|---|---|---|
| Polymer additive | PMSA | 14-16% wt | PMSA | 4-6% wt | PMSA | 14-16% wt |
| FSA additive | Alkyl | 0.13-0.16 mol % | Alkyl | 0.01-0.03 mol % | Amine | 0.01-0.03 mol % |
| Buffer | Tris | | PBS | | PBS | |
| pH Range | | 7.3-7.5 | | 7.4-7.7 | | 7.4-7.7 |

| | | | |
|---|---|---|---|
| Kd (mM) | 0.36 | 2.2 | — |
| [solution value] | [0.07] | [6] | |
| ΔF (enhancement | 2.93× | 2.36× | 2.53× |
| vs. solution) | [10 mM] | [100 mM] | [0.1 mM] |
| [sugar challenge] | | | |

The formulation optimization experiments described above used Design-Expert 6.0.5 (Stat-Ease, Inc., Minneapolis, Minn.) to design several Design of Experiments (DOE's). Among other variables in formulation which were optimized in each DOE were buffer type (HEPES, PBS and Tris) and pH (from 6.6 to 7.8). Surprisingly, the optimal formulation constituents and concentration ranges were quite different for each protein. In all cases, however, substantial performance improvements were obtained for the optimized formulations in comparison to either solution performance or performance in unmodified sol-gels.

### Example 7

### Entrapment of Mutant GGBP in UV-Crosslinked Hydrogel

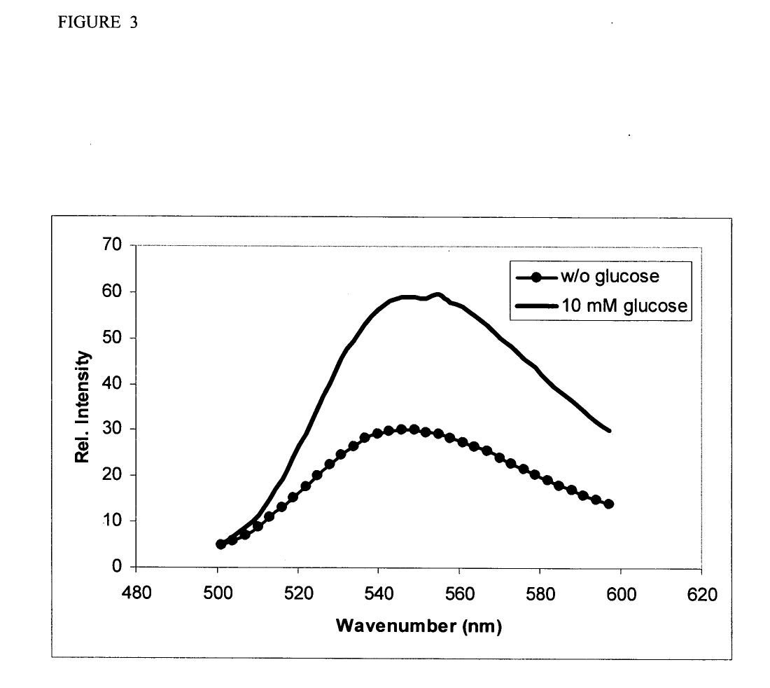

This example describes the entrapment of GGBP H152C in UV cross-linked hydrogel matrix and the effect of the matrix on the fluorescence change and binding affinity. In this experiment SbQ-PVA from Polysciences Inc. was added 100 ul of PBS buffer and mixed for one hour to mix in a rotary mixer. 80 ul of this solution was then mixed with 20 ul of labeled protein. Final protein concentration was spectroscopically determined to be 0.15 mg/ml. After mixing, aliquots were dispensed into 96-well plates and dried in a chamber maintained at 20% humidity for 12 h followed by curing with UV light. Wells containing protein encapsulated in matrix were challenge with 2 ul of 10 mM glucose and compared to protein solution without matrix having equivalent protein loading. FIG. 3 shows the ability of the mutated protein matrix to respond to the analyte in a manner, and with a sensitivity, equivalent to that obtained in solution. The Kd of the entrapped protein was comparable to that obtained in solution.

### Example 8

### Immobilization of Binding Protein within a Dialysis Membrane

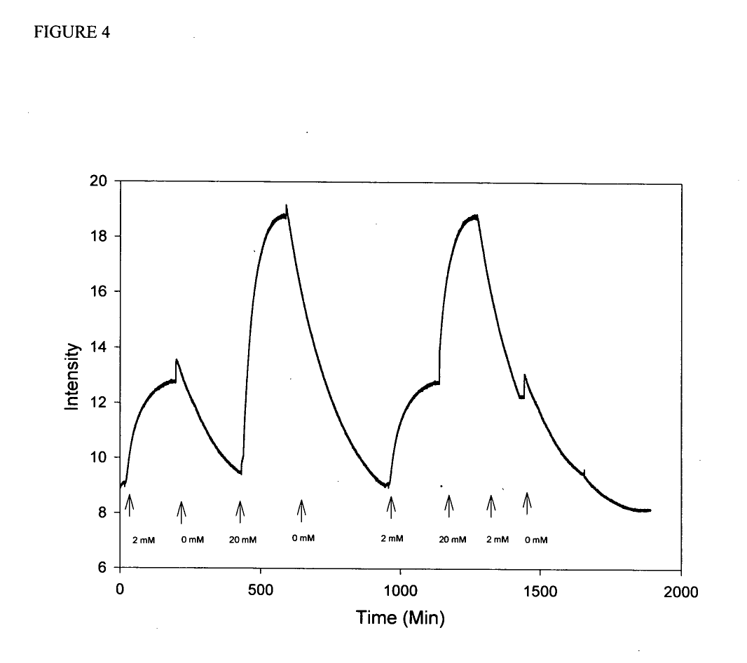

This example describes the immobilization of a biosensor of the instant invention into a dialysis membrane matrix and the ability of the matrix to provide reversible and continuous readings. Using a Varian Eclipse fluorimeter with a fiber optic attachment, GGBP L238C/A213C protein (2 μM in PBS buffer) entrapped within a dialysis membrane having a molecular cut-off of 3500 Daltons affixed to the distal end of the fiber. Solutions were prepared containing PBS buffer, 2 mM, and 20 mM glucose in PBS buffer. With the probe in PBS solution, readings were recorded at 0.02 seconds intervals of the emission wavelength 521 nm, followed by insertion of the fiber into the glucose solutions. Replacement of the fiber into buffer-only solution resulted in the return of initial signal. FIG. 4 depicts multiple cycles alternating between buffer and glucose solutions demonstrating the reversibility of the biosensor entrapped within a permeable matrix within physiological range. Similar results were observed with sol-gel entrapped samples demonstrating applicability for continuous use.

### Example 9

### Preparation of a Hydrogel Glucose Biosensor by One Pot Method

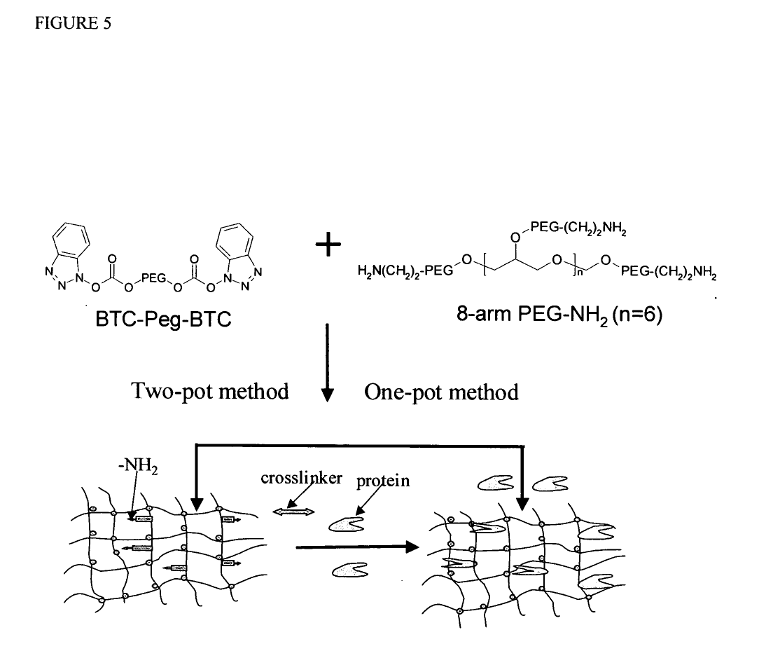

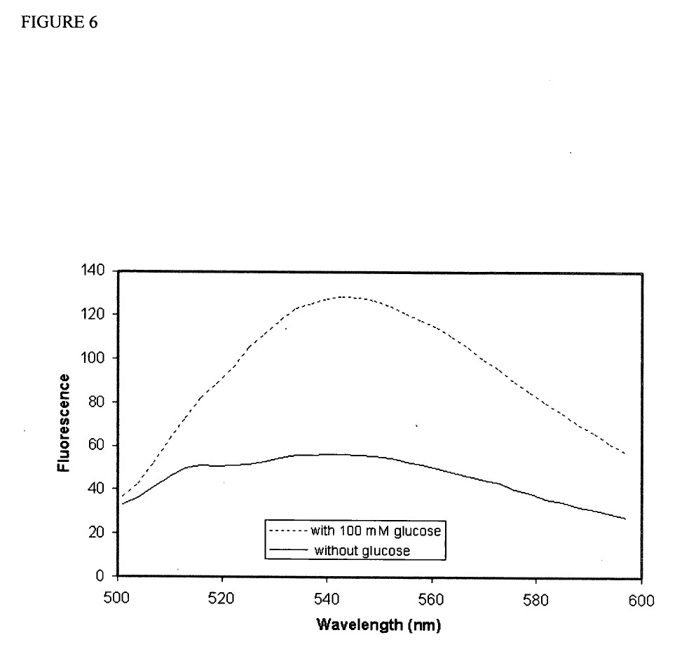

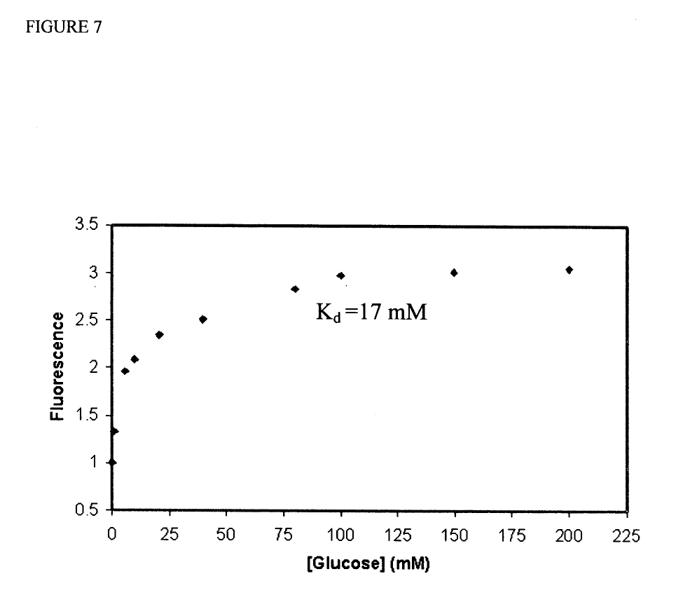

This example describes the coupling of a binding molecule to the hydrogel matrix during the hydrogel formation. Prior to hydrogel synthesis, the GGBP mutant E149C/A213R/L238S was labeled with N-((2-(iodoacetoxy)ethyl)-N-methyl)amino-7-nitrobenz-2-oxa-1,3-diazole (IANBD) (Molecular Probes, Eugene, Oreg.) and purified to provide a protein concentration of 52.7 μM, and a dye/protein ratio 1.37. An aliquot of 25.7 mg of 8-arm PEG-NH₂ (10,000 MW, Nektar Therapeutics, Huntsville, Ala.) was dissolved in 250 μL of PBS buffer (pH 7.4) in a 1.5 mL Eppendorf vial, and 250 μL of the above NBD-labeled GGBP in PBS buffer was added. The mixture was vortexed before and after adding 24.5 mg of PEG-bis-benzotriazolyl carbonate (BTC-PEG-BTC, 3400 MW, Nektar Therapeutics, Huntsville, Ala.) in 500 μL PBS buffer (pH 7.4) and was injected immediately between two glass plates separated by a 2 mm spacer. Hydrogel formation was completed in approximately 5 minutes. The glass plates were clamped together and the reaction was continued at room temperature for at least two hours. During the reaction, the benzotriazolyl carbonate groups from BTC-PEG-BTC react with both the amino groups of the 8-arm PEG-NH₂ and the amino groups of GGBP, forming carbamate bonds. FIG. 5 illustrates the reaction scheme for hydrogel formation and protein immobilization. After the reaction was complete, the formed hydrogel sheet was punched into 5 mm diameter disks with a dermal biopsy punch. All the disks were then soaked in PBS buffer (pH 7.4) for two days to wash away any unreacted monomer and protein. The glucose response of the formed hydrogel disks was tested in a 96 well plate using a Varian Cary Eclipse fluorometer with an excitation wavelength of 475 nm and emission scanned between 500 and 600 nm. The results are shown in FIG. 6. FIG. 7 illustrates the changes in fluorescence response to glucose concentration of a representative sample of hydrogel disks prepared by the methods of the present invention. The fluorescence increased with the increase of glucose concentration in the range of 0 to 100 mM glucose. The glucose binding constant K_{d} for the hydrogel biosensor was calculated as about 17 mM, which is similar to the binding constant of the free binding protein towards glucose in solution (12 mM). The K_{d} was determined from the following relationships
F=F_{inf}+(F_{inf}−F₀)/(x/(x/K_{d}))

Where F is fluorescence intensity, F_{inf} is fluorescence at infinite glucose concentration, F₀ is fluorescence at zero glucose, and x is the glucose concentration.

### Example 10

### Preparation of a Hydrogel Glucose Biosensor by the Two Pot Method

This example describes the coupling of the binding molecule to blank hydrogel disks. The immobilization chemistry is also illustrated in FIG. 5. The hydrogel was prepared first in absence of binding protein with excess of amino groups. The binding protein was coupled to the hydrogel matrix through a bifunctional crosslinker. A typical example of this two pot system is illustrated as follows: 100.3 mg of 8-arm PEG-NH₂ was dissolved in 2 mL PBS buffer (pH 7.4) in a 15 mL polypropylene conical tube and was mixed with 98.2 mg BTC-PEG-BTC in 1.96 mL PBS buffer (pH 7.4). The mixture was vortexed and injected between two glass plates separated by a 2 mm spacer. The glass plates were clamped together and the reaction was continued at room temperature for at least two hours. After the reaction was complete, the hydrogel sheet was punched into 5 mm diameter disks using a dermal biopsy punch. The blank disks were soaked in 30 mL PBS (pH 7.4) for two days to wash away any monomer residuals. To begin the coupling of the binding proteins, blank disks were removed from the PBS and soaked in a 1.7 mg/mL solution of bis-(sulfosuccinimidyl) suberate (BS³, Pierce Biotechnology, Inc., Rockford, Ill.) in PBS buffer in a 2 mL cryogenic vial (Nalge, Inc., Rochester, N.Y.). This reaction was continued for 15 minutes at room temperature. The disks were then taken out and rinsed briefly with PBS buffer, and soaked in 200 μL of NBD-labeled E149C/A213R/L238S GGBP protein (83.4 μM with a dye/protein ratio of 1.3). The GGBP protein was allowed to couple to the hydrogel matrix for at least two hours. All the disks were then soaked in PBS buffer (pH 7.4) for two days to wash away unattached proteins.

The glucose responsiveness of the hydrogel disks was tested. The hydrogel biosensors were placed in the wells of a black 96 well plate along with 180 μL PBS buffer per disk, and the initial fluorescence intensities were measured using a CytoFluor fluorescence multi-well plate reader (excitation and emission filters were centered at 485 nm and 530 nm, respectively). Next, 20 μL of 1 M glucose /water solution was added into each well, providing a final glucose concentration of 100 mM. The fluorescence intensity changes were recorded again after the solution was equilibrated for 20 minutes to allow glucose to completely diffuse into the hydrogel disks and bind with GGBP. Here, and in the following examples, the protein binding response is defined as a change in fluorescence intensity, QF, which is the ratio of the fluorescence intensity of the hydrogel biosensor disks in the presence of 100 mM glucose concentration to the fluorescence intensity of the hydrogel biosensor disks in the absence of glucose. The obtained average QF of all hydrogel disks was 3.4±0.1.

### Example 11

### Coupling Binding Protein to Hydrogel through Site Specific Attachment

In the preceding two examples, the binding protein was coupled to the hydrogel randomly and primarily through one or more lysine sites of the protein. This example describes the selective attachment of a binding protein to a hydrogel through thiol groups at cysteine sites of the protein. Six PEG blank disks were prepared by the method described Example 2. These blank disks were soaked in I mL PBS buffer (pH 7.4) in a 2 mL cryogenic vial. Next, 4.9 mg of solid Sulfo-LC-SPDP (Pierce Biotechnology, Inc) was added to the mixture, and the reaction was continued at room temperature for one hour. The disks were removed, washed in PBS buffer for about 3.5 hours and then soaked overnight in a solution of 200 μL single NBD-labeled E149C/A213C/L238S GGBP (protein concentration 14.8 μM with dye/protein ratio 1.1, predominately labeled at E149C). The glucose response of the hydrogel biosensors was then tested after they were washed in PBS for two days.

In the above procedure, the PEG hydrogel matrix, with excess amine groups, was coupled through reaction with the N-hydroxysuccinimidyl groups of sulfo-LC-SPDP. Disulfide bonds were then formed with between the thiol of the free cysteine residues of the protein and the 2-pyridyl disulfide residues of sulfo-LC-SPDP. This procedure provides an attachment method for selectively and uniformly coupling binding molecules within a hydrogel matrix. Table 5 compares the glucose binding response of the binding proteins coupled to the hydrogel using the above-described procedures with the binding response of the free protein. Also included in Table 5 are additional hydrogel biosensors prepared using procedures described herein. The protein binding responses of the hydrogel disks are comparable to that of the corresponding proteins in solution, suggesting that the selective coupling through specific sites allows protein conformational freedom.

TABLE 5
QF of Various Mutant Binding Proteins

| | | | | PEG-1NBD- | | PEG-2NBD- |
|---|---|---|---|---|---|---|
| | 1NBD- | PEG-1NBD- | 1NBD- | E149C/A213 | 2NBD- | E149C/ |
| | E149C/A213 | E149C/A213 | E149C/A213 | R/L238C | E149C/A213C/ | A213C/L238C |
| | C/L238S | C/L238S specific | R/L238C | specific | L238C | specific |
| Sample | solution | attachment | solution | attachment | solution | attachment |
| QF | 4.2 ± 0.1 | 5.4 ± 0.3 | 2.3 ± 0.8 | 2.8 ± 0.1 | 2.8 | 2.4 ± 0.4 |

### Example 12

### Effect of the Binding Protein Concentration and Cross-Linker to Polymer Ratio on the Glucose Binding Fluorescence Response of the Hydrogel Biosensor

Hydrogel biosensors comprising varying amounts of NBD-labeled E149C/A213R/L238S GGBP were prepared by the methods illustrated in Example 1 and were tested for their glucose response using a CytoFluor fluorescence multi-well plate reader. Table 6 displays the binding response of the hydrogel biosensors over a range of concentrations of binding proteins. Over an intial protein concentration range, from about 4 to about 25 μM, the binding responses are very similar, suggesting that the immobilized binding proteins were able to undergo glucose-induced conformational changes, regardless of the concentration of the binding protein.

TABLE 6
QF of Biosensors with Varying Concentrations of Binding Protein

| Initial protein concentration (μM) | QF |
|---|---|
| 4 | 5.4 ± 0.6 |
| 7 | 5.7 ± 0.8 |
| 11.2 | 4.3 ± 0.1 |
| 20 | 5.9 ± 0.1 |
| 25 | 5.8 ± 0.2 |

Various formulations of the hydrogel biosensors were also tested. Specifically, biosensors with various binding protein concentrations and various polymer/cross-linker ratios were assessed. As shown in Table 7, a QF of up to 7.8 was attainable, which approaches the QF of about 10 that is obtained for free protein upon binding 100 mM glucose in solution.

TABLE 7
QF values of Various Formulations of Hydrogels

| Polymer Concn. | NH₂/BTC Ratio | F₀ | QF |
|---|---|---|---|
| 10 | 3 | 1072 | 7.8 |
| 10 | 2 | 1293 | 6.4 |
| 8 | 2.5 | 1423 | 6.3 |
| 15 | 2.5 | 460 | 5.8 |
| 15 | 2.5 | 811 | 5.5 |
| 15 | 3.2 | 941 | 4.5 |
| 15 | 2.5 | 1257 | 3.7 |
| 20 | 2 | 374 | 2.5 |
| 15 | 1.8 | 326 | 2.5 |
| 20 | 3 | 674 | 2.3 |
| 25 | 2.5 | 207 | 2.1 |

### Example 13

### Stability of the Hydrogel Glucose Sensor

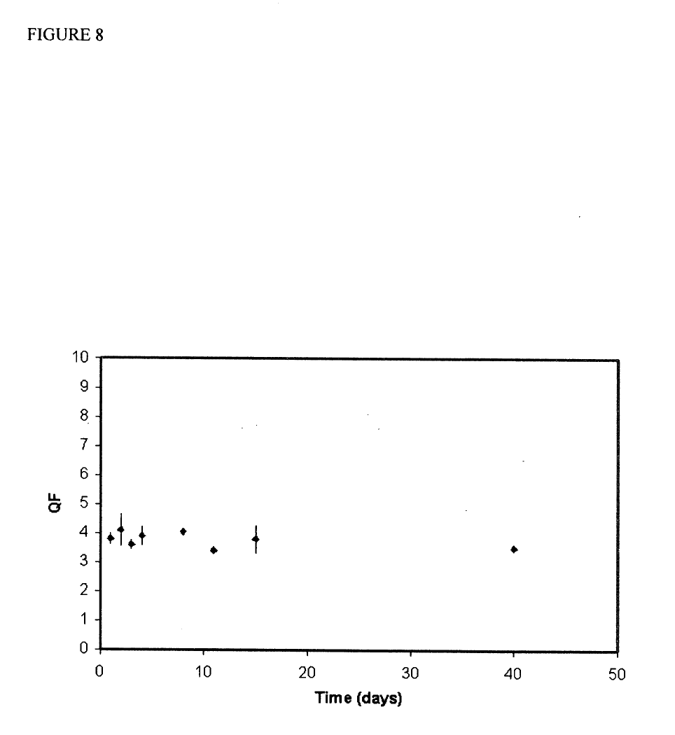

This example illustrates the stability of hydrogel disks prepared by the method of Example 1. Thirty hydrogel disks with immobilized NBD-labeled GGBP were stored in 20 mL PBS solution (pH 7.4) at room temperature. At varying time intervals, the hydrogel biosensor disks were taken out and the glucose binding response (QF) was determined. The results are depicted in FIG. 8. The hydrogel disks showed a stable glucose response for more than one month.

### Example 14

### Binding Protein Immobilized in Hydrogel Crosslinked by 6-arm PEG-NH₂ with BTC-PEG-BTC

This example illustrates that other multi-arm PEG amines, in addition to 8-arm PEG-NH₂, can be crosslinked into a hydrogel and used to covalently immobilize a binding protein. A solution of 40 mg of 6-arm PEG-NH₂ (10,000 MW, Sunbio Inc, Korea) in 300 μL of PBS buffer (pH 7.4) in a 1.5 mL Eppendorf vial was mixed with 100 μL of NBD-labeled E149C/A213R/L238S GGBP in PBS buffer (protein concentration 15.7 μM with dye/protein ratio 1). Next, 18.3 mg of PEG-bis-benzotriazolyl carbonate (BTC-PEG-BTC, 3400 mw, Nektar therapeutics, Huntsville, Ala.) in 183 μL PBS buffer (pH 7.4) was added, and the solution mixture was vortexed and immediately injected between two glass plates separated by a 2 mm spacer. The hydrogel formed within 1 minute. The glass plates were clamped together, and the reaction continued at room temperature for at least two hours. After the reaction was complete, the formed hydrogel sheet was punched into 5 mm diameter disks using a dermal biopsy punch. All the disks were soaked in PBS buffer (pH 7.4) for two days to wash away any unreacted monomer and unattached protein. The glucose response of all disks was tested by CytoFluor fluorescence multi-well plate reader. The obtained average QF was 3.1±0.4.

### Example 15

### Binding Protein Immobilized in Hydrogel Crosslinked by 8-arm PEG-NH₂ with BS³

This example illustrates that binding molecules can also be covalently immobilized in a hydrogel crosslinked by 8-arm PEG-NH₂, using non-PEG bifunctional crosslinkers. A solution of 60 mg of 8-arm PEG-NH₂ (10,000 MW, from Nektar therapeutics, Huntsville, Ala.) in 300 μL of MES buffer (pH 6.5) in a 1.5 mL Eppendorf vial was prepared and mixed with 100 μL of NBD-labeled E149C/A213R/L238S GGBP in MES buffer (protein concentration 86.5 μM, with dye/protein ratio 1.1). To this was added 13.8 mg of BS³ (Bis-(sulfosuccinimidyl) suberate (Pierce) in 92 μL MES buffer. The solution mixture was vortexed and injected immediately between two glass plates separated by a 2 mm spacer. The hydrogel formed within 1 minute. The glass plates were clamped together, and the reaction continued at room temperature for at least two hours. After the reaction was done, the formed hydrogel sheet was punched into 5 mm diameter disks using a dermal biopsy punch. All the disks were soaked in PBS buffer (pH 7.4) for two days to wash away any unreacted monomer and unattached protein. The glucose response (QF) of all disks was tested using a CytoFluor fluorescence multi-well plate reader. The average QF was 4.2±1.4.

### Example 16

### Hydrogel Glucose Biosensor on Optical Fiber

This example illustrates the use of a hydrogel with binding protein coated on an optical fiber as a device for continuous monitoring glucose concentration in vitro and in vivo. A solution of 25.7 mg of 8-arm PEG-NH₂ in 0.3 mL PBS buffer (pH 7.4) in a 1.5 mL Eppendorf vial was mixed with 200 μL of NBD-labeled E149C/A23 1R/L238S GGBP in PBS buffer (protein concentration is 125.5 μM with dye/protein ratio 0.9). Next, 24.5 mg of BTC-PEG-BTC in 0.5 mL PBS buffer was added to the mixture. After thorough mixing, the final mixture was manually coated onto the end of a 470 nm optical fiber (Ceram Optec, East Longmeadow, Mass.), and the reaction was allowed to continue for at least two hours. The gel formed within a few minutes and formed very thin hydrogel films with a thickness of about 100 μm to about 500 μm on the optical fiber tip. Because PEG is a hydrophilic polymer, it can form strong hydrogen bonds with the hydroxyl groups on the surface of the silica core of the fiber tip.

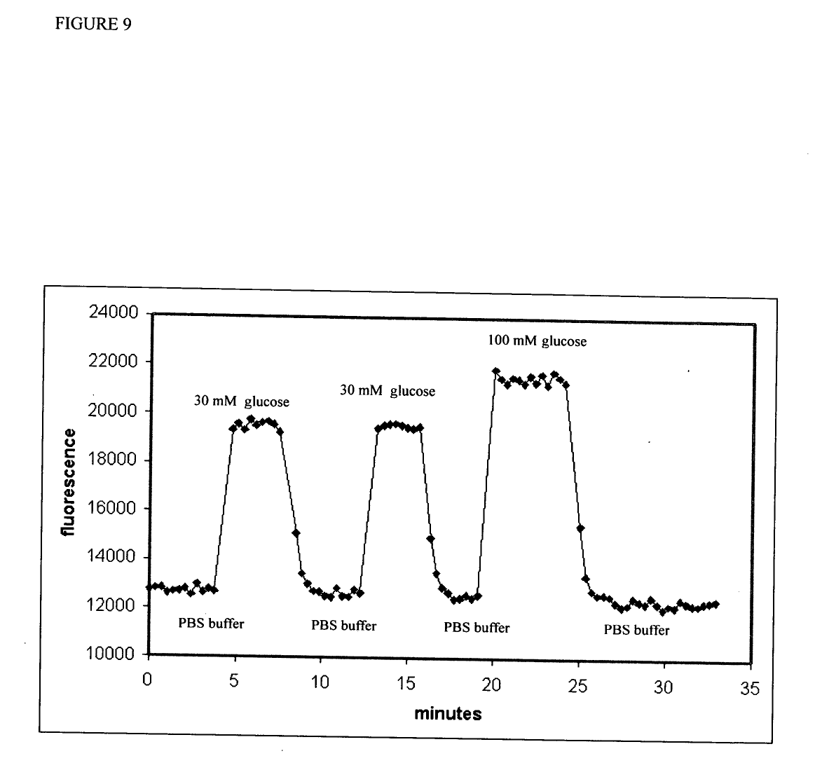

The hydrogel biosensor was used to continuously monitor glucose concentration changes using a custom fluorometer. An example of a fluorometer is described in U.S. application Ser. No. 10/721,797, filed Nov. 26, 2003, which is hereby incorporated by reference. The fluorometer was equipped with a 470 nm LED light source and a dichroic filter to reflect the 470 nm excitation towards the input end of the fiber and to transmit the fluorescence from the fiber towards a 550 nm bandpass filter leading to a single photon counting photomultiplier tube detector. Glass aspheric lenses were used both for beam collimation and to focus light into the fibers and onto the detectors. FIG. 9 depicts the fluorescence response of the fiber optic sensor following its immersion into solutions of the indicated glucose concentrations (0, 30, and 100 mM glucose). Due to the thinness of the biosensor, glucose was able to permeate to the hydrogel matrix quickly, and the sensor reached an apparent equilibrium within approximately one minute, demonstrating that the sensor can be used to monitor glucose concentration changes in real time.

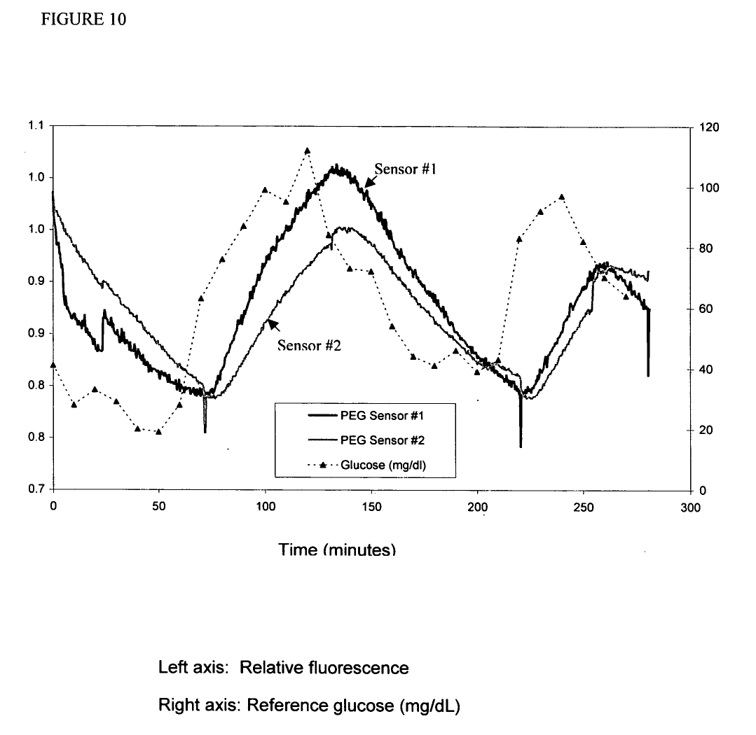

Additional hydrogel biosensors were fabricated using the general procedures described above, except that the optical fibers were glued inside 21 gauge needles, and hydrogels were coated on the fiber tips to completely fill the needle bevels. The sensors were used to track in vivo glucose concentration changes in a pig. Two fiber optic sensors were inserted into the side of an anesthetized pig. Alternating solutions of lactated ringer's solution, with and without 10% dextrose, were infused through the ear vein of the pig to increase and decrease glucose levels in a controllable fashion. At ten minute intervals, blood samples were pulled from the vena cava of the pig through a chest catheter, and blood sugar readings were tested on a handheld blood glucose meter. The fluorescence intensity of the two biosensors was observed to track changing blood glucose levels in the anesthetized pig as shown in FIG. 10.

### Example 17

### Fatty acid Binding Protein Immobilized in a PEG Hydrogel

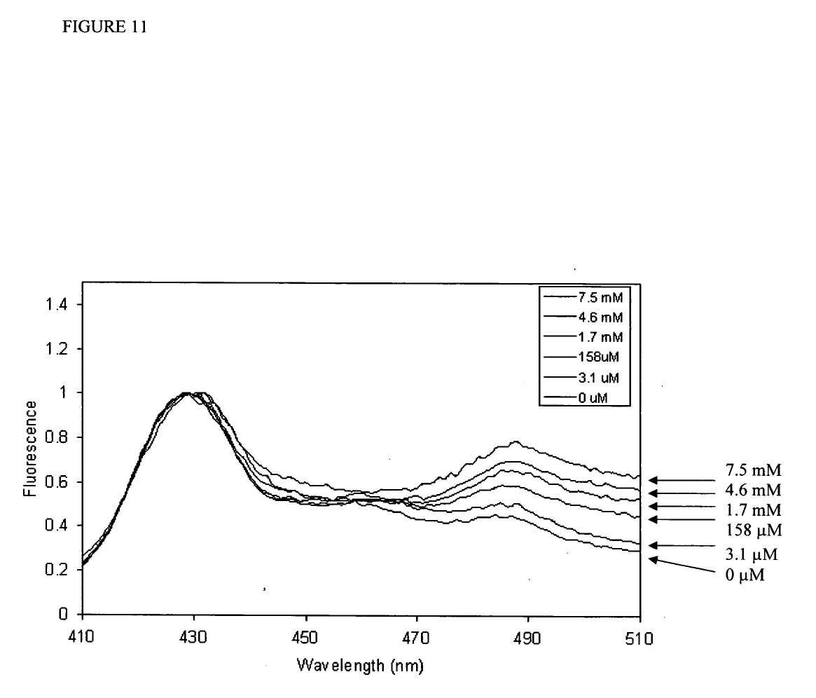

This example describes making hydrogel biosensors for fatty acid detection. A solution of 200 μg of ADIFAB (AcryloDated Intestinal Fatty Acid Binding Protein with dye/protein ratio approximately 1.0, Molecular Probes) in 1.0 mL of buffer (50 mM Tris, 1 mM EDTA, 0.05% azide, pH 8.0) was prepared. The binding protein solution (210 μL) was combined with 21 mg of 8-arm PEG-NH₂ (10,000 MW, Nektar) in a 1.5 mL Eppendorf vial. The mixture of 8-arm PEG-NH₂ and binding protein was further mixed with 18 mg of BTC-PEG-BTC (3,400 MW, Nektar) in 180 μL PBS buffer (pH 7.4) and vortexed. The mixture was immediately injected between two glass plates separated by a 2 mm spacer. After the reaction was complete, the formed hydrogel sheet was punched into 5 mm diameter disks, which were then soaked in PBS buffer for two days to wash away unbound protein and monomer residuals. The binding of fatty acid to the hydrogel disks was measured using a Varian Cary Eclipse fluorometer and 96 well plates (excitation was at 390 nm). FIG. 11 depicts the fluorescence response of the hydrogel disks to a wide range of arachadonic acid concentrations. The hydrogel sensor responded to FA (fatty acid, e.g., arachadonic acid) with a shift of fluorescence emission wavelength from 432 nm to 486 nm. Increasing FA concentration caused an increase in, the emission intensity at 486 nm.

### Example 18

### Preparation of a Hydrogel Maltose Biosensor by the One-Pot Method

Prior to hydrogel synthesis, the MBP mutant S337C was labeled with N-((2-(iodoacetoxy)ethyl)-N-methyl)amino-7-nitrobenz-2-oxa-1,3-diazole (IANBD) (according to the procedure of Gilardi, et al., Anal Chem. 66:3840-7 (1994)) and purified to provide a protein concentration of 13.8 μM, and a dye/protein ratio of 2. An aliquot of 30 mg of 6-arm PEG-NH₂ (10,000 MW, Sunbio Inc., Orinda, Calif.) was dissolved in 300 μL of the above NBD-labeled MBP in PBS buffer. The mixture was vortexed before and after adding 13.5 mg of BTC-PEG-BTC in 135 μL PBS buffer (pH 7.4) and was injected immediately between two glass plates separated by a 1 mm spacer. Hydrogel formation was complete in approximately 5 minutes. The glass plates were clamped together and the reaction was continued at room temperature for at least two hours. During the reaction, the benzotriazolyl carbonate groups from BTC-PEG-BTC react with amino groups of the 6-arm PEG-NH₂ and the amino groups of MBP, forming carbamate bonds. After the reaction was complete, the formed hydrogel sheet was punched into 4 mm diameter disks with a dermal biopsy punch. All the disks were then soaked in PBS buffer (pH 7.4) for two days to wash away any unreacted monomer and protein.

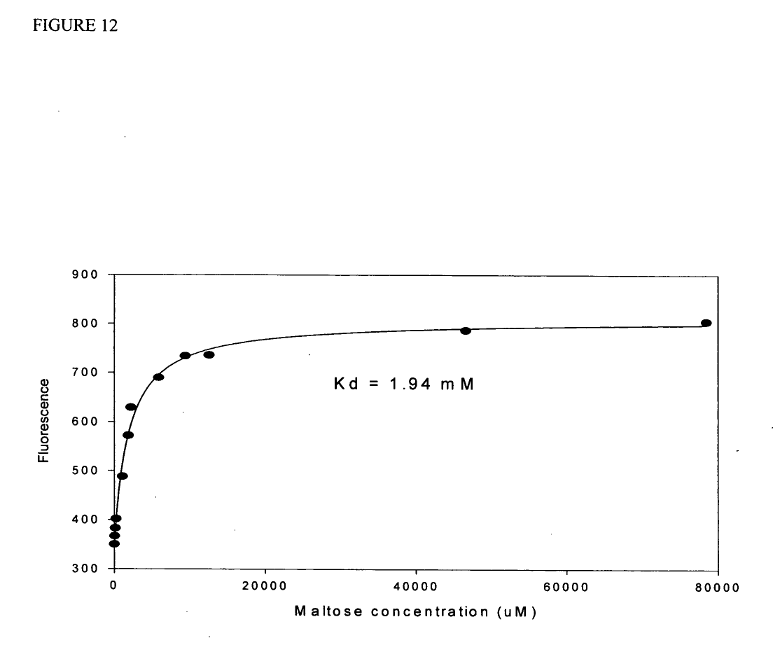

The maltose response of the formed hydrogel disk was tested in a 96 well plate in the presence of different maltose concentrations using a Varian Cary Eclipse fluorometer with an excitation wavelength of 475 nm and emission scanned between 500 and 600 nm. The fluorescence intensity at wavelength of 540 nm was read. The results are shown in FIG. 12, which demonstrates that fluorescence increased with the increase of maltose concentration in the range of 0 to about 12.6 mM maltose. The maltose binding constant Kd for the hydrogel biosensor was calculated to be 1.94 mM, using the methods of calculation described in Example 9. In comparison, the Kd of the same mutant maltose binding protein is 0.062 mM [Gilardi]. Surprisingly, the encapsulation or entrapment of binding protein caused a shift of more than an order of magnitude in Kd compared to solution.

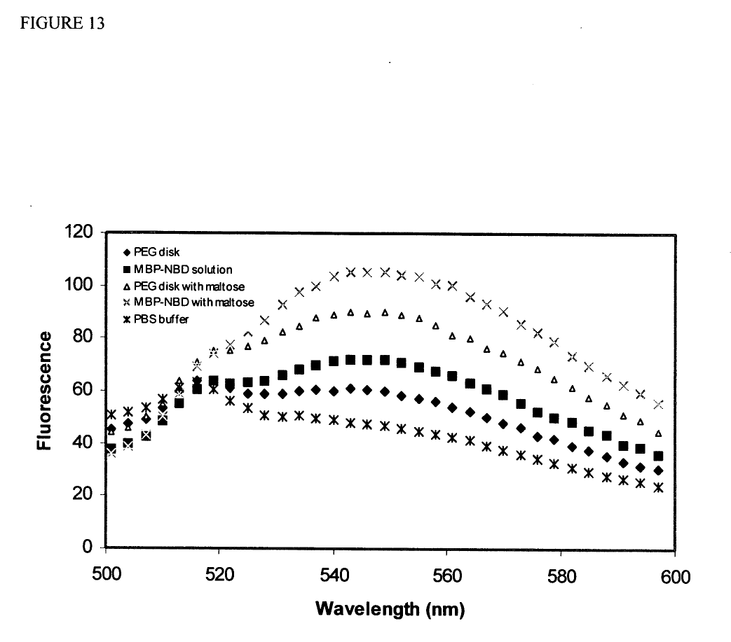

The QF of hydrogel biosensors was also measured. The hydrogel biosensors were placed in the wells of a white 96 well plate along with 180 μL PBS buffer per disk, and the initial fluorescence intensities were measured using a Varian Cary Eclipse fluorometer with an excitation wavelength of 475 nm and emission scanned between 500 and 600 nm. Next, 20 μL of 1 M maltose/water solution was added into each well, providing a final maltose concentration of 100 mM. The fluorescence intensity changes were recorded again after the solution was equilibrated for 20 minutes to allow maltose to completely diffuse into the hydrogel disks and bind with MBP. FIG. 13 shows that the QF of the maltose-binding hydrogels, as defined as it was previously herein, was about 4, whereas the QF of the MBP-NBD in solution was about 3.

### Example 19

### Preparation of a Hydrogel Maltose Biosensor by the Two-Pot Method

The hydrogel was prepared first in absence of binding protein with excess of amino groups. The binding protein was coupled to the hydrogel matrix through a bifunctional crosslinker. A typical example of this two pot system is illustrated as follows: 30 mg of 6-arm PEG-NH₂ was dissolved in 300 μL PBS buffer (pH 7.4) in a 1.5 mL Eppendorf vial and was mixed with 13.5 mg BTC-PEG-BTC in 135 μL PBS buffer (pH 7.4). The mixture was vortexed and injected between two glass plates separated by a 1 mm spacer. The glass plates were clamped together and the reaction was continued at room temperature for at least two hours. After the reaction was complete, the hydrogel sheet was punched into 1 mm diameter disks using a dermal biopsy punch. The blank disks were soaked in 30 mL PBS (pH 7.4) for two days to wash away any monomer residuals. To couple the binding proteins to the blank hydrogel disks, blank disks were removed from the PBS and soaked in a 1.7 mg/mL solution of BS³ in PBS buffer in a 2 mL cryogenic vial. This reaction was continued for 15 minutes at room temperature. The disks were then taken out and rinsed briefly with PBS buffer, and soaked in 200 μL of NBD-labeled S337C MBP protein (13.2 μM with a dye/protein ratio of 2). The MBP protein was allowed to couple to the hydrogel matrix for at least two hours. All of the disks were then soaked in PBS buffer (pH 7.4) for two days to extract unattached proteins.

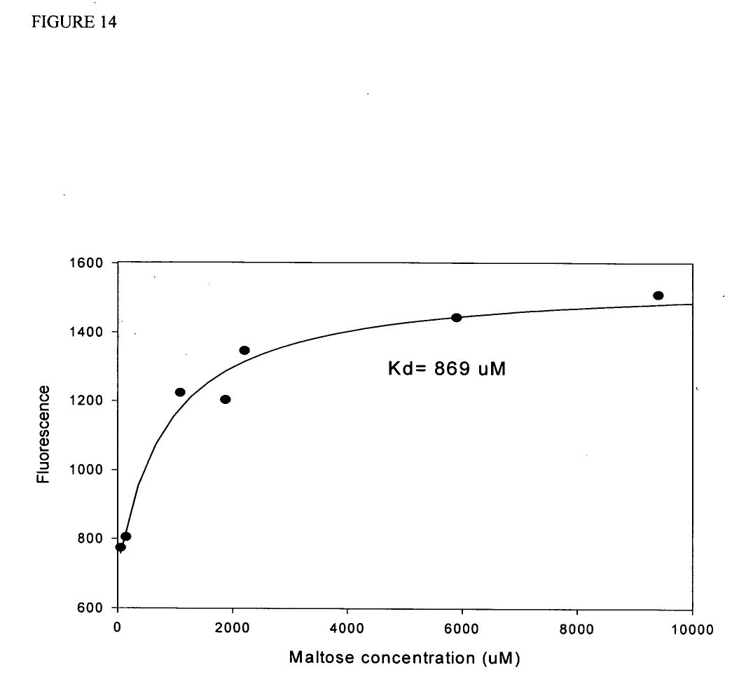

The maltose response of the hydrogel disks, prepared according to the two-pot method, was tested in the presence of different maltose concentrations in a 96 well plate using a Varian Cary Eclipse fluorometer with an excitation wavelength of 475 nm and emission scanned between 500 and 600 nm. The fluorescence intensity at wavelength of 540 nm was read. The results are shown in FIG. 14, which demonstrates that fluorescence increased with the increase of maltose concentration in the range of 0 to about 9.4 mM maltose. The maltose binding constant K_{d} for the MBP hydrogel biosensor prepared by the two-pot method was calculated to be about 0.869 mM, using the methods of K_{d} calculation described in Example 9. Again, the encapsulation or entrapment of binding protein caused an unexpected shift of more than an order of magnitude in Kd compared to solution.

### Example 20

### Hydrogel Maltose Biosensor on Optical Fiber by the One-Pot Method

A solution of 30 mg of 6-arm PEG-NH₂ in 300 ul NBD-labeled S337C MBP in PBS buffer (protein concentration is 13.8 μM with dye/protein ratio 2). Next, 13.5 mg of BTC-PEG-BTC in 135 ul PBS buffer was added to the mixture. After thorough mixing, the final mixture was manually coated onto the end of a 470 um optical fiber, and the reaction was allowed to continue for at least two hours. The gel formed within a few minutes and formed very thin hydrogel films with a thickness of about 100 μm to about 500 μm on the optical fiber tip.

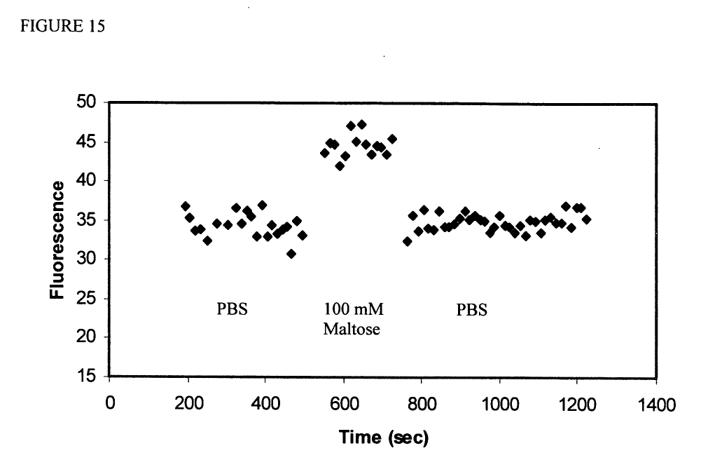

The hydrogel biosensor was used to continuously monitor maltose concentration changes using a S2000 Miniature Fiber Optic Spectrometer (Ocean Optics, Dunedin, Fla.). FIG. 15 depicts the fluorescence response of the fiber optic sensor following its immersion into alternating solutions of PBS and maltose at a concentration of 100 mM. The optical fiber responded to changes in maltose concentrations within 13 seconds.

### Example 21

### Preparation of HEMA-MAA Hydrogels For Protein Immobilization

2-Hydroxyethyl methacrylate (HEMA) was purchased from Polysciences, Inc, Warrington, Pa., and methacrylic acid (MAA) was purchased from Aldrich, Milwaukee, Wis. Triethylene glycol dimethacrylate (TEGDMA) and polyethylene glycol (400) dimethacrylate (PEGDMA) were purchased from Sartomer, Exton, Pa. 1-Ethyl-3-(3-dimethylaminopropyl)carbodiimide hydrochloride (EDC), N-hydroxysuccinimide (NHS), benzoyl peroxide (BPO) and dimethylformamide (DMF) were purchased from Aldrich, Milwaukee, Wis. In the following examples, swelling was determined by measuring the hydrogel disk volume changes before and after being soaked in water for one day. The disk volumes were measured by placing them in a graduated cylinder and measuring displaced water volume.

One gram of HEMA (7.7 mmol), 74 mg MAA (0.86 mmol), 24 mg TEGDMA (0.08 mmol) and 21 mg of BPO (0.087 mmol) were mixed together in a 20 ml scintillation vial, followed by the addition of 0.47 ml water. The molar ratio of HEMA:MAA was about 9:1, and the total monomer weight concentration (HEMA, MAA, TEGDMA) was about 70%. The mixture was deoxygenated by argon gas purging for about 5 minutes. The solution was the transferred to 1 ml polypropylene syringes and capped. The syringes were incubated at 70° C. for about 90 minutes. Upon cooling, cylinder-shape hydrogels were removed from the syringes and sectioned into disks of about 4 mm diameter by 2 mm thickness. The disks were soaked in distilled water for two days to remove low molecular weight impurities. Additional examples of hydrogels prepared in accordance with the above described method are summarized in Table 8.

### Example 22

### Entrapment of Binding Protein in HEMA-MAA Hydrogel

Following the procedure of Example 21, various formulations of the HEMA-MAA hydrogels were synthesized using different HEMA/MAA molar ratio, crosslinker contents, crosslinker types, and reaction times. Prior to hydrogel synthesis, NBD labeled GBP stock solution was prepared. For example, 1 mg of IANBD labeled GBP was dissolved in 2.5 ml 0.1 MES buffer (pH 6.5). The hydrogel disks were soaked overnight in aqueous solutions containing the mutant GGBP-NBD. The hydrogel biosensors were placed in the wells of a black 96 well plate along with 180 μL PBS buffer per disk, and the initial fluorescence intensities were measured using a CytoFluor fluorescence multi-well plate reader (excitation and emission filters were centered at 485 nm and 530 nm, respectively). Next, 20 μL of 1 M glucose /water solution was added into each well, providing a final glucose concentration of 100 mM. The fluorescence intensity changes were recorded again after the solution was equilibrated for 20 minutes to allow glucose to completely diffuse into the hydrogel disks and bind with GGBP. Table 8 demonstrates that the poly(HEMA) hydrogel glucose biosensors prepared by the method described were responsive to glucose. Table 8 further summarizes the determined swelling data of different HEMA-MAA hydrogel formulations synthesized in aqueous phase.

TABLE 8
QF and Swelling Data of HEMA-MAA Hydrogel Glucose Biosensors

| | | | | | | swelling | | |
|---|---|---|---|---|---|---|---|---|
| | Monomer | HEMA:MAA | Crosslinker | | Polym. | (vol. | mean | std |
| No. | Conc. (%) | molar ratio | (%) | Crosslinker | Time | increase. %) | QF | dev |
| 1 | 70 | 90/10 | 1 | TEGDMA | 90 min | 36 | 4.8 | 0.5 |
| 2 | 90 | 90/10 | 4 | TEGDMA | 90 min | 56 | 2.1 | 0.5 |
| 3 | 70 | 90/10 | 4 | PEGDMA | 90 min | 29 | 2.4 | 0.8 |
| 4 | 90 | 90/10 | 1 | PEGDMA | 90 min | 64 | 3.6 | 0.6 |
| 5 | 90 | 99/1 | 4 | PEGDMA | 90 min | 76 | 1.8 | 0.1 |
| 6 | 70 | 99/1 | 1 | PEGDMA | 90 min | 41 | 3.4 | 0.7 |
| 7 | 70 | 99/1 | 4 | TEGDMA | 90 min | 30 | 3.1 | 2.2 |
| 8 | 90 | 99/1 | 1 | TEGDMA | 90 min | 55 | 2.2 | 0.8 |
| 9 | 70 | 90/10 | 1 | TEGDMA | overnight | 11 | 4.1 | 0.3 |
| 10 | 90 | 90/10 | 4 | TEGDMA | overnight | 39 | 3.6 | 1.6 |
| 11 | 70 | 90/10 | 4 | PEGDMA | overnight | 15 | 2.0 | 0.4 |
| 12 | 00 | 90/10 | 1 | PEGDMA | overnight | 37 | 3.1 | 0.9 |
| 13 | 90 | 99/1 | 4 | PEGDMA | overnight | 54 | 1.4 | 0.3 |
| 14 | 70 | 99/1 | 1 | PEGDMA | overnight | 39 | 2.0 | 0.2 |
| 15 | 70 | 99/1 | 4 | TEGDMA | overnight | 25 | 2.0 | 0.2 |
| 16 | 90 | 99/1 | 1 | TEGDMA | overnight | 56 | 1.9 | 0.6 |

### Example 23

### Covalently Immobilized Binding Protein in Aqueous Phase Synthesized HEMA-MAA Hydrogels

This example describes the coupling of a binding protein to the HEMA-MAA copolymer hydrogel as prepared in Example 21. Hydrogel disks as prepared in Example 21 were exposed to a bulk solution of 0.5mg mutant GGBP in 300 μl of 0.1 M MES buffer solution and stored refrigerated overnight. A sample of disks and bulk solution (200 ul) was mixed with an equal volume ratio of EDC/NHS mixture. Various concentrations of EDC were tested for the protein coupling reaction, with the EDC/NHS ration being held constant at 4:1. The protein-hydrogel matrix coupling was continued at room temperature for 4 hours. The disks were removed and exposed to ethanolamine (1M, pH 8.5) for 1 hour to quench the coupling reaction. The disk were then soaked in 0.1 M PBS buffer for 3 to 5 days to remove unattached protein. At an EDC concentration of 5 mM, the average measured QF of the resultant protein-coupled hydrogel disks was about 2.5±0.2, demonstrating that the hydrogel glucose biosensors were repsonsive to glucose. The QFs of hydrogels prepared at the various concentrations of EDC are listed in Table 9.

TABLE 9
Effect of Various EDC concentrations on QF of
Covalently Immobilized Protein

| EDC Concentration (mM) | QF |
|---|---|
| 2.6 | 3.2 ± 0.3 |
| 5 | 2.5 ± 0.2 |
| 10 | 2.6 ± 0.2 |
| 15 | 2.3 ± 0.4 |
| 30 | 1.9 ± 0.3 |
| 75 | 1.2 ± 0.1 |

### Example 24

### Organic Solvent Phase Synthesis Method for HEMA-MAA Hydrogels

A solution of 0.5 g HEMA (3.9 mmol), 37 mg MAA (0.43 mmol), 24 mg PEGDMA (0.04 mmol) and 11 mg of BPO (0.04 mmol) were mixed together in a 20 ml scintillation vial, followed by the addition of 2.2 g DMF. The HEMA:MAA molar ratio was 9:1 respectively, and the total monomer weight concentration (HEMA, MAA, PEGDMA) was 20%. The mixture was purged with argon gas for 5 minutes. The solution was transferred to 1 ml polypropylene syringes and capped. The syringes were put in incubated at 70° C. overnight. The syringes were then removed and the hydrogel was cut into disks of about 4 mm diameter and 2 mm thickness. The disks were swelled in DMF for 12 hours and then soaked in water until the hydrogel turned opaque. This DMF-water extraction process was repeated until the hydrogel disks remained transparent when exposed to water. The resultant disks were then used for protein immobilization.

### Example 25

### Covalently Immobilized Binding Protein to HEMA-MAA Hydrogel Synthesized in Organic Phase

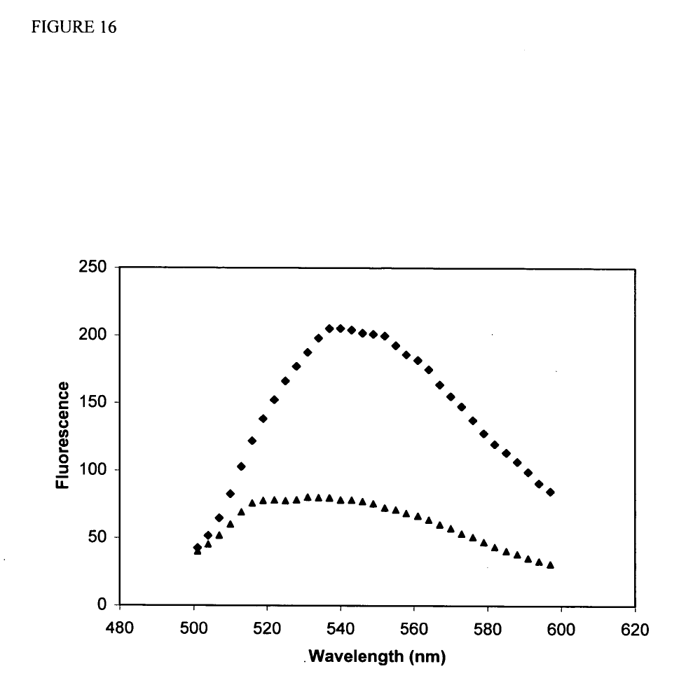

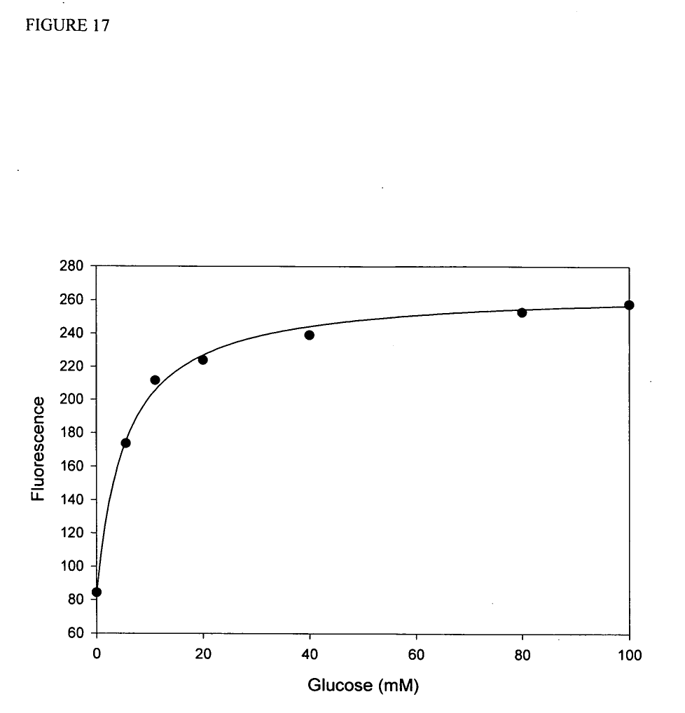

Following the procedure as essentially described in Example 23, mutant GGBP-NBD was covalently immobilized within the HEMA-MAA hydrogels of the previous example, which were prepared in organic solvent phase. The glucose response of the formed hydrogel disks was tested in a 96 well plate using a Varian Cary Eclipse fluorometer with an excitation wavelength of 475 nm and emission scanned between 500 and 600 nm. As shown in FIG. 16 and FIG. 17 the hydrogel glucose biosensor, prepared according to the procedures herein, provided a functional biosensor having a QF of about 4.1. Further, changes in fluorescence of the hydrogel glucose biosensor correlated to changes in glucose concentrations in solution. The fluorescence increased with the increase of glucose concentration in the range of 0 to 100 mM glucose. The glucose binding constant Kd for the hydrogel glucose biosensor was calculated to be about 5 mM, using methods previously described herein.

### Example 26

### The Effect of the Cross-Linker and Monomer Concentration, and Monomer Ratio on Fluorescence Response of the Hydrogel Biosensor

Hydrogel biosensors comprising varying amounts of monomer concentration, HEMA/MAA molar ratios, and crosslinking agents were prepared in organic solvent by the methods illustrated in Example 24 and the protein was covalently immobilized in hydrogels by methods illustrated in Example 23. The glucose responses of the hydrogel biosensors were tested using a Varian Cary Eclipse fluorometer as taught in the previous examples herein. Table 10 shows the binding response of hydrogel biosensors over a range of different monomer concentrations, MAA molar ratios, and crosslinker contents. For example, hydrogel glucose biosensor prepared according to the methods of the present invention with a monomer concentration of 20% have higher QF values than hydrogels prepared with monomer concentration of 50%.

TABLE 10
QF Values of Hydrogel Glucose Biosensors Prepared
under Various Conditions

| | | Mono- | | | | |
|---|---|---|---|---|---|---|
| | | mer | | | | |
| Exp. | Crosslinker | Conc. | | HEMA:MAA | Mean | |
| no | (mol %) | (wt. %) | Crosslinker | molar ratio | QF | Std |
| 1 | 2 | 20 | PEGMA | 90/10 | 6 | 0.1 |
| 2 | 2 | 50 | TEGDMA | 90/10 | 3.6 | 0.3 |
| 3 | 2 | 50 | PEGMA | 90/10 | 4 | 0.8 |
| 4 | 1 | 20 | PEGMA | 95/5 | 5.5 | 0.1 |
| 5 | 1 | 20 | PEGMA | 90/10 | 6 | 0.8 |
| 6 | 1 | 50 | PEGMA | 95/5 | 3 | 0.5 |
| 7 | 2 | 50 | PEGMA | 95/5 | 3.9 | 0.1 |
| 8 | 1 | 50 | PEGMA | 90/10 | 5.8 | 0.2 |
| 9 | 2 | 50 | TEGDMA | 95/5 | 3.7 | 0.1 |
| 10 | 1 | 50 | TEGDMA | 90/10 | 5.3 | 0.7 |
| 11 | 1 | 50 | TEGDMA | 95/5 | 3.5 | 0.4 |
| 12 | 2 | 20 | PEGMA | 95/5 | 4.4 | 0.1 |

### Example 27

### Effect of Protein Loading, EDC Concentration, and Coupling Time to Organic Phase Synthesized HEMA-MAA Hydrogels

This example describes how protein loading concentration, EDC concentration and reaction time affect the glucose response of hydrogel glucose biosensors. The HEMA-MAA hydrogel was prepared similarly to experiment no. 1 in Example 26 and exposed to different concentrations of GGBP-NBD in 0.1 M MES buffer overnight. The hydrogel disks, in the GGBP-NBD/MES solution, were then soaked with equal volumes of EDC/NHS solution mixture (10-30 mM EDC, EDC/NHS concentration ratio 4:1). The protein-hydrogel coupling reaction was continued for 2-4 hours at room temperature. The disks were then exposed to ethanolamine (1M, pH 8.5) for 0.5 hour to quench the protein-hydrogel coupling reaction. All the disks were then soaked in 0.1 M PBS buffer for at least 3 days to remove unattached protein. The QF of the disks was measured with a Varian Cary Eclipse fluorometer as described in previous examples herein. Table 11 summarizes the experimental results. As shown in Table 11, a QF of up to 8.6 was attained, which is a value that approaches the QF for free protein (QF ˜10) in a 100 mM glucose solution.

TABLE 11
QF Values of Hydrogel Glucose Biosensors
Prepared under Various Conditions

| | EDC | | | | |
|---|---|---|---|---|---|
| Expt. | Concentration | Protein | Reaction | Mean | |
| No. | (mM) | Concentration (μM) | Time (h) | QF | STd |
| 1 | 30 | 30 | 4 | 5.1 | 0.2 |
| 2 | 10 | 30 | 4 | 5.9 | 0.2 |
| 3 | 30 | 10 | 4 | 5.7 | 0.2 |
| 4 | 20 | 20 | 3 | 5.7 | 0.4 |
| 5 | 10 | 10 | 2 | 7 | 1.1 |
| 6 | 10 | 30 | 2 | 5.8 | 0.2 |
| 7 | 10 | 10 | 4 | 8.6 | 0.4 |
| 8 | 20 | 20 | 3 | 5.7 | 0.3 |
| 9 | 30 | 10 | 2 | 6.1 | 0.5 |
| 10 | 20 | 20 | 3 | 5.8 | 0.4 |
| 11 | 30 | 30 | 2 | 5.7 | 0.2 |

### Example 28

### Stability of the Hydrogel Glucose Biosensors

This example illustrates the stability of hydrogel disks prepared by the methods of Examples 21 and 24. Two hydrogel disks with immobilized GGBP-NBD were stored in 20 mL PBS solution (pH 7.4) at room temperature. At varying time intervals, glucose binding response (QF) was determined, and the results are depicted in Table 12.

TABLE 12
Stability of Hydrogel Glucose Biosensors

| Time (days) | Mean QF | Std |
|---|---|---|
| 4 | 5.3 | 0.7 |
| 12 | 4.7 | 0.4 |
| 25 | 5.9 | 0.1 |

### Example 29

### Optical Fiber Coupled HEMA-MAA Hydrogel Biosensor and Continuous In Vivo Testing

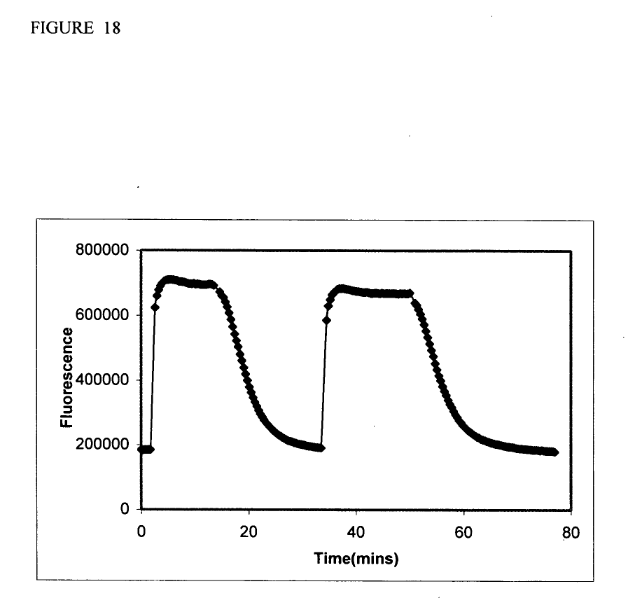

A solution of 0.5 g HEMA (3.9 mmol), 37 mg MAA (0.43 mmol), 24 mg PEGDMA (0.04 mmol) and 11 mg of BPO (0.04 mmol) were mixed together in a 20 ml scintillation vial, and 2.2 g DMF was then added, followed by argon purging. The polymerization solution was then transferred into 1 ml polypropylene syringe having attached thereto a 470 nm optical fiber (Cream Optec, East Longmeadow, Mass.) glued into the 21 gauge needle (Loctite 4011 (Loctite, Rockyhill, Conn.)). The solution was polymerized at 70° C. overnight. After cooling to room temperature the fiber/needle bevel was removed from the syringe. The optical fiber tip contained a thin hydrogel film having a thickness of about 100 to about 500 μm. The matrix was washed overnight with water (200 μL) in a capped vial. The fiber was then inserted into 50 μl of GGBP-NBD solution (18 μM protein concentration, dye/protein ratio was 1.1) in 0.1 M MES buffer and the labeled protein was allowed to infuse for 2 hours. A mixture of 50 μl of EDC (10 mM) and NHS (2.5 mM) was then added to the protein/fiber solution, and the coupling reaction was allowed to proceed for 4 hours before being quenched by addition of 100 μl ethanolamine (1 M) for 30 minutes. The resultant biosensor probe was then soaked in 200 μl PBS buffer for two days before use/testing. Monitoring changes in glucose concentration on a continuing basis using the fiber optic-hydrogel biosensor was achieved using a fluorometer. The fluorometer comprised a 470 nm LED light source having a dichroic filter to reflect the 470 nm excitation towards the input end of the fiber. The transmitted fluorescence from the fiber tip containing the hydrogel was directed through a 550 nm bandpass filter. A single photon counting photomultiplier tube detector was used. Glass aspheric lenses were used both for beam collimation and to focus light into the fibers and onto the detector. Continuous monitoring of glucose concentration changes over time using the device herein described is shown graphically in FIG. 18. These results demonstrate the feasibility of continuously monitoring glucose concentration changes by monitoring changes in fluorescence of the hydrogel glucose biosensor affixed within a fiber optic device.

The hydrogel glucose biosensors described herein were also used to track in vivo glucose concentration changes in anesthetized Yorkshire swine pigs. The pigs were sedated using a mixture of Telazol, Ketamine, and Rompin. An endotracheal tube was inserted and the pig was given Isofluorane to maintain anesthesia. A chest catheter was placed in either the vena cava or the carotid artery for blood sampling and an IV catheter was placed in the ear for maintenance fluid delivery, glucose delivery and control.

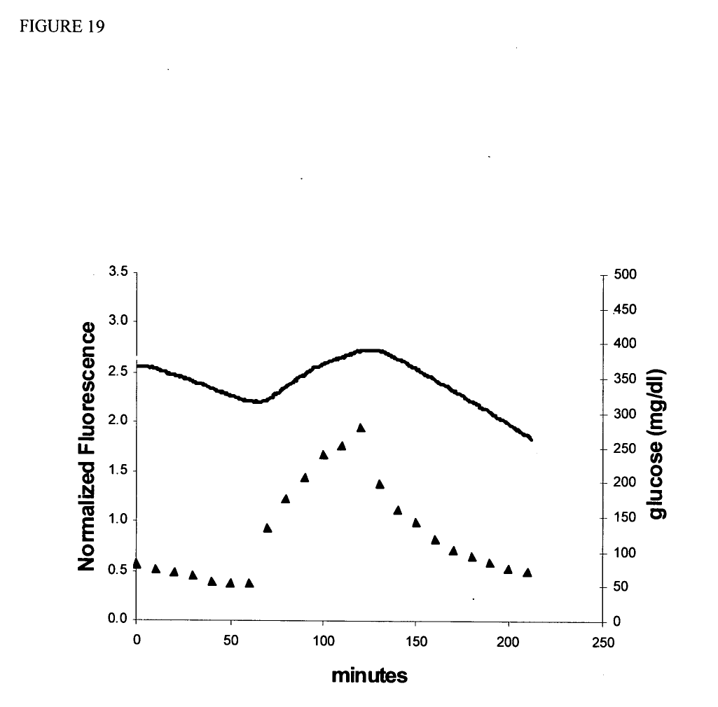

Optical fiber-hydrogel glucose biosensors, prepared as described above, in 21 gauge needles, were inserted intradermally, bevel down, and taped securely. A surface skin probe was taped on skin within 1 inch of the sensors to monitor temperature. Sampling via the sensor with simultaneously blood sampling was begun and continued at 10 minute intervals. Alternating solutions of lactated ringer's solution, with and without 10% dextrose, were infused through the ear vein of the pig to increase and decrease glucose levels in a controllable fashion. The blood samples were obtained from the vena cava of the pig through a chest catheter, and blood sugar readings were tested on a handheld blood glucose meter. When data collection was complete, the sensors were removed and placed in buffer solution. FIG. 19 depicts the changes in fluorescence intensity of the hydrogel glucose biosensors in response to changing blood glucose levels in the glucose-controlled anesthetized pig. This data demonstrates, among other attributes, the reversible and continuous monitoring utility of the optical fiber-hydrogel sensor described herein.

### Example 30

### Hydrogel Glucose Biosensor Formation using Photo Crosslinking

Prior to hydrogel synthesis, mutant GGBP E149C/A213R/L238S was labeled with IANBD and purified to provide a protein concentration of 10.4 μM, and a dye/protein ratio 1.0. To prepare the hydrogel, 200 μL of poly(ethylene glycol) dimethacrylate 1000 (PEGDMA) (Degussa) was mixed with 2.5 μL of 6-arm PEG with acrylate terminations (Biolink Life Sciences, Cary, N.C., USA), 1 μL of 2-Hydroxy-2-methylpropiophenone (Sigma) as a photo initiator, 8.25 mg of sorbitol (Sigma) and 450 μL of PBS. The mixture was vortexed for about 15 seconds and 150 μL of GGBP-NBD was then added to the solution. The mixture was then injected in between two glass plates separated by a 1 mm spacer and exposed to UV light (450 watts at 22 cm) for 3.5 minutes. After the reaction was complete, the hydrogel sheets were punched into 5 mm diameter disks with a dermal biopsy punch.

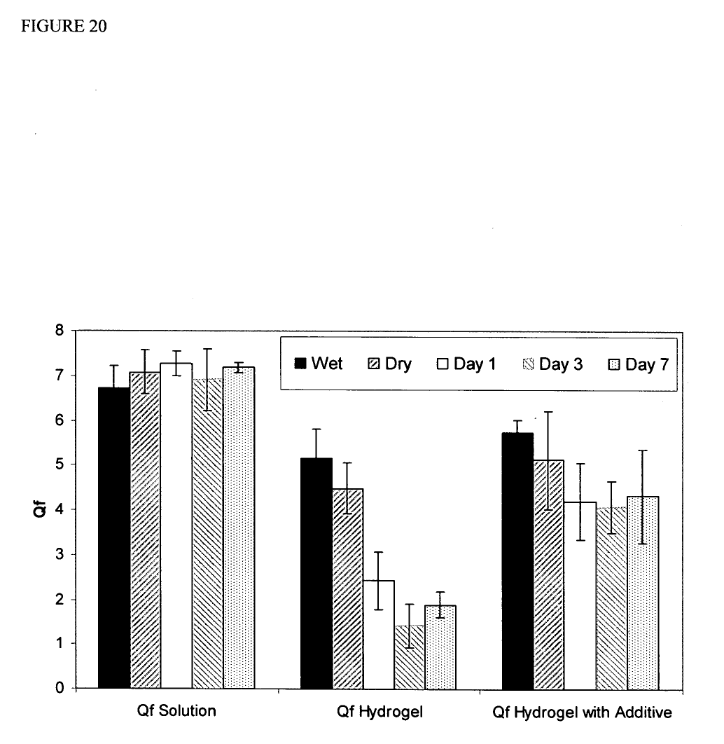

The responsiveness of the hydrogel glucose biosensors, as measured by QF, was assessed. Fluorescence was measured using a CytoFluor fluorescence multi-well plate reader (excitation and emission filters were centered at 485 nm and 530 nm, respectively). The hydrogel biosensors were placed in the wells of a black 96 well plate along with 180 μL PBS buffer per disk and fluorescence was measured. Next, 20 μL of 1 M glucose /water solution was added into each well, providing a final glucose concentration of 100 mM and fluorescence intensity changes were recorded again after the solution was equilibrated for 20 minutes to allow glucose to completely diffuse into the hydrogel disks and bind with GGBP. FIG. 20 demonstrates that hydrogel glucose biosensors prepared by the photo crosslinking methods described herein were responsive to glucose. FIG. 20 also shows that the hydrogels were stable for at least 7 days after formation and that the hydrogels were responsive both before and after lyophilization and rehydration.

### Example 31

### Functional Derivatization of Binding Protein by Reaction with Reactive Groups

Providing acrylate functionality is but one example of derivatizing a fluorescent labeled binding protein with a reactive moiety. Thus, to a solution of 0.33 g of N-acryloxy succidimide (Aldrich Chemicals) in 4mL of PBS at pH of 7 was added 1 mL of a 1 mg/mL of E149C/A213R/L238S fluorescent labeled mutant binding protein. A ratio of 10 parts N-acryloxy succidimide to 1 parts protein was used in this experiment. After two hours, the reaction mixture was eluted through a NAP 10 column with collection of the second fraction. The collected derivatized binding protein was used in subsequent hydrogel polymerization reactions as described infra. It is understood that ratios of reactive moiety to protein can be optimally varied so long as the resultant derivatized protein maintains its binding functionality before or after polymerization.

### Example 32

### Hydrogel Formation by Polymerization of Functionally Derivatized Binding Protein via Photo Crosslinking

To a solution of 1 mL of polydimethylsiloxane end-terminated with methyacryloxypropyl functionality (DMS-R18, Gelest, inhibitor removed using Inhibitor Remover Column 30631.2, Aldrich) and 20 μL of 2-hydroxy-2-methypropiophenone (photo initiator) was added 100 μL (PBS buffer, 6.9 pH) of acryloyl derivatized-fluorescently labeled binding protein. This solution was placed between glass microscope slides separated with 1 mm spacers and exposed to UV light for 3.5 minutes to provided a hydrogel. From this, 4 mm by 1 mm diameter disks were prepared using a biopsy punch. Glucose response and Qf values were obtained and compared to solution values of non-derivatized protein. Qf values of the derivatized protein co-polymerized into the hydrogel were about 85% of a non-derivatized protein in solution. Thus, derivatizing and polymerizing the protein resulted in no substantial loss of protein functionality or fluorescence by reporter group.

1. A biosensor comprising
a) a crosslinked polymeric hydrogel; and
b) a binding molecule, wherein said binding molecule is covalently attached to said hydrogel, and wherein said binding molecule is capable of generating a detectable signal directly upon binding of a target molecule to said binding molecule.

2. The composition of claim 1, wherein said crosslinked polymeric hydrogel comprises a polymer selected from the group consisting of poly(ethylene glycol) (PEG), poly(hydroxyethyl methacrylate) (poly(HEMA)) and copolymers thereof.

3. The composition of claim 2, wherein said polymer is PEG.

4. The composition of claim 2, wherein said polymer is poly(HEMA).

5. The composition of claim 3, wherein said binding molecule is a protein.

6. The binding protein of claim 5, wherein said protein is selected from the group consisting of galactose/glucose binding protein (GGBP), maltose binding protein (MBP), ribose binding protein (RBP), arabinose binding protein (ABP), dipeptide binding protein (DPBP), glutamine binding protein (QBP), iron binding protein (FeBP), histidine binding protein (HBP), phosphate binding protein (PhosBP), oligopeptide binding protein (OppA), fatty acid binding protein (FABP) and derivatives thereof.

7. The composition of claim 6, wherein said binding protein is galactose/glucose binding protein (GGBP).

8. The composition of claim 7, wherein said galactose/glucose binding protein comprises a fluorescent reporter group.

9. The composition of claim 6, wherein said binding protein is maltose binding protein.

10. The composition of claim 9, wherein said maltose binding protein comprises a fluorescent reporter group.

11. A method of making a biosensor, said biosensor comprising a crosslinked polymeric hydrogel and a binding molecule, said binding molecule being covalently attached to said hydrogel and capable of generating a detectable signal directly upon binding of a target molecule to said binding molecule, said method comprising polymerizing and crosslinking monomers in the presence of water and said binding molecule.

12. The method of claim 11, wherein said crosslinked polymeric hydrogel comprises a polymer selected from the group consisting of poly(ethylene glycol) (PEG), poly(hydroxyethyl methacrylate) (poly(HEMA)) and copolymers thereof.

13. The composition of claim 12, wherein said polymer is PEG.

14. The composition of claim 12, wherein said polymer is poly(HEMA).

15. The method of claim 13, wherein said binding molecule is a protein.

16. The binding protein of claim 15, wherein said binding protein is selected from the group consisting of galactose/glucose binding protein (GGBP), maltose binding protein (MBP), ribose binding protein (RBP), arabinose binding protein (ABP), dipeptide binding protein (DPBP), glutamine binding protein (QBP), iron binding protein (FeBP), histidine binding protein (HBP), phosphate binding protein (PhosBP), oligopeptide binding protein (OppA), fatty acid binding protein (FABP) and derivatives thereof.

17. The method of claim 16, wherein said binding protein is galactose/glucose binding protein.

18. The method of claim 17, wherein said galactose/glucose binding protein comprises a fluorescent reporter group.

19. The method of claim 16, wherein said binding protein is maltose binding protein.

20. The method of claim 19, wherein said maltose binding protein comprises a fluorescent reporter group.

21. A method of detecting an analyte in a sample comprising
a) providing a biosensor comprising a crosslinked, polymeric hydrogel and a binding molecule, said binding molecule being covalently attached to said hydrogel and being capable of generating a detectable signal directly upon binding of said analyte to said binding molecule
b) contacting said biosensor with said sample
c) comparing the signal generated by said binding molecule when said biosensor is contacted with said sample with the signal generated by said binding molecule when said biosensor is contacted with an analyte-free control sample, wherein a difference in the signal generated by said binding molecule when said biosensor is contacted with said test sample, as compared to when said biosensor is contacted with said control sample, indicates that test sample contains the analyte.

22. The method of claim 21, wherein said sample is biological fluid.

23. The method of claim 22, wherein said fluid is selected from the group consisting of interstitial fluid and blood.

24. The method of claim 23, wherein said analyte is glucose.

25. A method of making a polymeric hydrogel biosensor, said method comprising polymerizing a functionally derivatized binding protein and at least one monomer to produce said crosslinked polymeric hydrogel biosensor

26. The method of claim 25, wherein said polymerization comprises the use of UV light.

27. The method of claim 26, wherein said method further comprises addition of an additive to said functionally derivatized binding protein and monomer.

28. The method of claim 27, wherein said additive is selected from the group consisting of a carbohydrate, polyol and saccharide.

29. The method of claim 28, wherein said additive is selected from the group consisting of trehalose, sorbitol, sucrose, dextran and PEG.

30. The method of claim 25, wherein said functionally derivatized binding protein comprises mutant GGBP with at least one covalently attached acrylate functional group.

31. The method of claim 30, wherein said acrylate functional groups are coupled to lysine residues of said mutant GGBP.

32. The method of claim 25, wherein said monomer is multi-functionalized.

33. The method of claim 32, wherein said monomer is functionalized with acrylate functional groups.

34. The method of claim 25, wherein said crosslinking comprises using a chemical.

35. The method of claim 34, wherein said chemical is peroxide.

36. A method of making a crosslinked polymeric hydrogel biosensor, said method comprising photo polymerizing a monomer in the presence of a binding protein to produce said polymeric hydrogel biosensor comprising said binding protein.

37. The method of claim 36, wherein said method further comprises addition of an additive to said binding protein and monomer.

38. The method of claim 37, wherein said additive is selected from the group consisting of a carbohydrate, polyol and saccharide.

39. The method of claim 38, wherein said additive is selected from the group consisting of trehalose, sorbitol, sucrose, dextran and PEG.

40. The method of claim 36, wherein said monomer is functionalized with acrylate functional groups.

41. A biosensor comprising a binding protein wherein said biosensor has an apparent dissociation constant (Kd) of at least one order of magnitude greater than the Kd of said free binding protein in solution.

42. The biosensor of claim 41, wherein said binding protein is selected from the group consisting of galactose/glucose binding protein (GGBP), maltose binding protein (MBP), ribose binding protein (RBP), arabinose binding protein (ABP), dipeptide binding protein (DPBP), glutamine binding protein (QBP), iron binding protein (FeBP), histidine binding protein (HBP), phosphate binding protein (PhosBP), oligopeptide binding protein (OppA), fatty acid binding protein (FABP) and derivatives thereof.

43. The method of claim 42, wherein said binding protein is galactose/glucose binding protein.

44. The method of claim 43, wherein said galactose/glucose binding protein comprises a fluorescent reporter group.
